(19) United States
(12) Patent Application Publication
(10) Pub. No.: US 2009/0092966 A1
(43) Pub. Date: Apr. 9, 2009

(54) COLLOIDAL METAL AGGREGATES AND METHODS OF USE

(75) Inventors: Jon P. Anderson, Lincoln, NE (US); Teresa M. Urlacher, Wahoo, NE (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LI-COR, Inc., Lincoln, NE (US)

(21) Appl. No.: 11/755,672

(22) Filed: May 30, 2007

Related U.S. Application Data
(60) Provisional application No. 60/803,591, filed on May 31, 2006.

(51) Int. Cl. C12Q 1/68; B01J 19/00; G01N 33/00; G01N 33/48; G01N 33/53; G01N 33/563; G01N 33/573

(52) U.S. Cl. 435/6; 422/241; 435/7.1; 435/7.4; 436/513; 436/63; 436/86

(57) ABSTRACT

Metal colloidal aggregates substrates useful for metal enhanced fluorescence applications, are disclosed. Method of making and using these colloidal aggregates for enhancing the fluorescent signal in biological assays are also described.

### CROSS-REFERENCE TO RELATED APPLICATIONS

This application claims priority to U.S. Application No. 60/803,591 filed May 31, 2006, the teaching of which is hereby incorporated by reference in its entirety for all purposes.

### GOVERNMENT RIGHTS

This invention was made with government support under 1 R43 RR021785-01 awarded by the National Institutes of Health. The government has certain rights in the invention.

### BACKGROUND OF THE INVENTION

Fluorescence is a highly sensitive and convenient method for detection that is widely used in biotechnology, genomics, immunoassays, array technologies, imaging, and drug discovery. Fluorescent molecules can easily be attached to a wide variety of target molecules, including DNA, RNA, antibodies, and proteins. Emissions generated from fluorophores predominately take place in free space, producing little interaction with surrounding molecules. When molecular interactions do take place in the local environment, they mainly affect nonradiative processes of the fluorophore, such as quenching. Therefore, traditionally, fluorophores that produce the highest quantum yields and thus the largest emissions, are often chosen as biological probes as they provide the highest detection sensitivity.

In order to improve the sensitivity and reliability of fluorescent groups, signal emissions from target molecules need to increase without increasing the signal from nonspecific molecules. It has been shown that when weakly emitting fluorophores are held in close proximity to metal surfaces (e.g., noble metal surfaces), a “metal enhanced fluorescence” (MEF) effect occurs wherein an increase in quantum yield and photostability, and a decrease in the fluorescence lifetime (i.e., an increase in radiative decay) of the fluorophore is observed. This dramatic increase in the fluorescence emission is seen in fluorophores that are held at a distance of roughly about 10 to about 2000 Å above metal surfaces. At a distance closer than about 50 Å of the metal surface, the metal will begin to quench the fluorophore emission. At a distance greater than about 2000 Å from the metal surface, the fluorophore displays free space characteristics. Preferably, a fluorophore is located roughly between about 25 to 1000 Å, more preferably between about 50 and 200 Å from the metal surface for optimal metal enhancement to take place. Depending on the distance of the fluorophore to the metal surface and the geometry of the metal surface, the enhancement of emission of the fluorophore can be up to 1000-fold (see, Wokaun, A., J. Chem. Phys., 1983, 79(1), 509-514; Holland, W. R., et al., Optics Letts., 1985, 10(8), 414-416; Glass, A. M., et al., Optics Letts., 1980, 5(9), 368-370). It is this region of enhancement that provides the greatest effect to a fluorophore's quantum yield and can aid in signal specificity, essentially amplifying only those fluorophores that are specifically located within this enhancement zone.

The MEF effect is derived from the interaction of the dipole moment of the fluorophore and the surface plasmon field of the metal resulting in an increase in the radiative decay rate, and stronger fluorescence emission. The result is that even weakly emitting materials (e.g., dyes, proteins, or DNA) with low quantum yields are transformed into more efficient fluorophores having shorter fluorescence lifetimes (see, Lakowicz, J. R. et al. Biochem. Biophys. Res. Comm., 2001, 286(5), 875-879). Additionally, the use of MEF on a periodic metal island surface may also allow for directional emission rather than isotropic photonic emissions, creating the opportunity to collect substantially more signal from the fluorophore (see, Lakowicz, J. R. et al., Photonics Spectra, 2001, 35, 96-104).

The inherent characteristics of MEF make it an appealing supplement to many commonly used fluorescent techniques, and give it the potential to be utilized in a wide range of applications. DNA, RNA, protein, antibody microarrays, along with fluorescent in situ hybridization (FISH) and immunoassays could directly benefit from the enhanced quantum yield, increased photostability, and decreased self-quenching using MEF.

Researchers have primarily focused on using either metal island films, or metal colloids as the metal surface for the enhancement of fluorescence, although other metal surfaces exist, e.g., nanorods, nano triangles, fractals, and the like. In particular, metal island films have been used for over two decades in Raman spectroscopy for signal enhancement and are now emerging in the field of fluorescence detection (see, Lakowicz, J. R., Anal. Biochem, 2001, 298, 1-24; Lakowicz, J. R., et al. Photonics Spectra, 2001, 35, 96-104; Lakowicz, J. R., et al., Anal. Biochem. 2002, 301, 261-277; Malicka, J., et al., Anal. Biochem, 2003, 317, 136-146; Malicka, J. et al., Anal. Chem., 2003, 75, 4408-4414; Malicka, J., et al., Biopolymers, 2003, 72, 96-104). Metal island films are composed of subwavelength sized patches of a highly conducting material located on an inert, typically solid, substrate such as glass. Randomly seeded metal islands that ranged in size from 20 nm to 500 nm have been successfully used to enhance a variety of fluorophores, although metal islands in the range of 30-80 nm are most common. (see, Lakowicz, J. R., et al., Anal. Biochem, 2002, 301, 261-267; Malicka, J. et al., Biopolymers, 2003, 72, 96-104; Malicka, J., J. Biomed Opt, 2003, 8, 472-478; Lakowicz, J. R., et al., Photochem Photobiol, 2003, 77, 604-607; Malicka, et al., Anal Biochem, 2003, 315, 57-66).

The challenges of MEF include the ability to reproducibly generate consistent metal structures. Currently, most metal island films are formed using silver either by chemical deposition from solution (dip-coating), or by evaporation of silver under high vacuum onto quartz or glass substrates (see, Lakowicz, J. R. Anal. Biochem, 2001, 298, 1-24). Also, extensive literature reports exist on the preparation of metal colloids suspensions, which are then deposited onto a substrate (see, Geddes, C. D., et al. J Phys Chem A, 2003, 107, 3443-3449). For example, colloid deposition onto glass slides is achieve by adding trisodium citrate solution to a warmed silver nitrate solution in a closed chamber on glass slides.

There have been many advances in the development of metal structures for MEF applications, however, many aspects concerning optimizing the properties of the metal surface for MEF remains unexplored. For example, research suggests that the size and the shape of the metal structures may play an important role in the ability to enhance the fluorescent signal (see, Gersten, J. I., et al. Surf Sci. 1985, 158, 165-189; Pugh, V. J., et al. Science, 2002, 298, 1759-1762). In view of the above, there remains a need in the art for new metal structure compositions that can optimize a fluorescent signal that will be useful in metal enhanced fluorescence application. Facile and reliable methods for making the metal structure compositions are also needed. The present invention fulfills these and other needs.

### BRIEF SUMMARY OF THE INVENTION

The present invention provides novel metal structures for metal enhanced fluorescence applications, useful in assay methods and systems. The metal structures are especially useful in biological applications.

As such, in one aspect, the present invention provides a system for detecting a specific binding pair, the system comprising: a substrate having a first member of a specific binding pair disposed thereon; and at least one metal colloidal aggregate proximate to the first binding member. The substrate is for example, glass, silanized glass, a quartz plate, plastic, a carbon nanotube, a metal or a membrane.

In another embodiment, the present invention provides a method for detecting a specific binding pair, comprising:
i) providing a substrate having at least one biomolecule disposed thereon and at least one metal colloidal aggregate proximate thereto;
ii) contacting the substrate with a first member of a specific binding pair wherein the first member of the specific binding pair attaches to the biomolecule;
iii) contacting the substrate with a second member of the specific binding pair; wherein the second member of a specific binding pair comprises at least one fluorescent group attached thereto; and
iv) irradiating the at least one fluorescent group with excitation radiation to detect the fluorescent group.

In yet another aspect, the present invention provides a method for detecting a specific binding pair, comprising:
i) providing a substrate having a first member of a specific binding pair disposed thereon and at least one metal colloidal aggregate proximate thereto;
ii) contacting the substrate with a second member of the specific binding pair, wherein the second member of a specific binding pair comprises at least one fluorescent group attached thereto; and
iii) irradiating the at least one fluorescent group with excitation radiation to detect fluorescence.

In another aspect, the present invention provides a method for detecting a specific binding pair, comprising:
i) providing a substrate having at least one biomolecule disposed thereon and at least one metal colloidal aggregate proximate thereto;
ii) contacting the substrate with a first member of a specific binding pair wherein the first member of the specific binding pair attaches to the biomolecule;
iii) contacting the substrate with a second member of the specific binding pair; wherein the second member of a specific binding pair comprises at least one enzyme group attached thereto; and
iv) incubating the substrate with a chemiluminescent compound to detect a luminescent product.

In still yet another aspect, the present invention provides a method for detecting a specific binding pair, comprising:
i) providing a substrate having a first member of a specific binding pair disposed thereon and at least one metal colloidal aggregate proximate thereto;
ii) contacting the substrate with a sample containing a second member of the specific binding pair, wherein the second member of a specific binding pair comprises at least one enzyme group attached thereto; and
iii) incubating the substrate with a chemiluminescent compound to detect a luminescent product.

In another embodiment, the present invention provides a two color ELISA (e.g., a near-infrared FLISA) method, comprising:
i) providing a substrate with at least two first specific binding members disposed thereon, wherein the at least two first specific binding members are the same or different;
ii) contacting the substrate with a sample containing at least two second specific binding members to form at least two specific bound pairs, wherein each of the at least two second specific binding members contain a first and a second fluorescent group, respectively; and
iii) irradiating the first and second fluorescent groups with excitation radiation to detect two color fluorescence.

The systems and methods of the present invention are particularly useful to detect a complimentary specific binding pair in an assay selected from a western blot, a northern blot, a Southern blot, cell-based assays, molecular beacon assays, protein arrays, DNA arrays and antibody arrays.

These and other objects, aspects and embodiments will become more apparent when read with the accompanying detailed description and drawings which follow.

### BRIEF DESCRIPTION OF THE DRAWINGS

FIGS. 1(a)-1(c) show scanning electron microscopy images at various magnifications of one embodiment of a metal colloidal aggregate on glass substrates.

FIG. 2(a)-2(f) show light microscope images of various metal surface compositions on a glass substrate. FIG. 2(a) shows an image of a silver island film on a plain glass substrate of the prior art. FIG. 2(b) shows a traditional colloid coated plain glass substrate surface of the prior art. FIG. 2(c) shows one embodiment of a silver metal colloidal aggregate on a plain glass substrate prepared by controlled aggregation. FIG. 2(d) shows an image of silver metal colloidal aggregate on a silaniated glass substrate prepared by controlled aggregation. FIG. 2(e) shows concentrated colloids on a glass substrate not prepared by controlled aggregation. FIG. 2(f) shows of one embodiment of a concentrated colloid on a glass substrate prepared by controlled aggregation.

FIG. 3 shows one embodiment of the present invention.

FIGS. 4(a)-4(c) show embodiments of the present invention.

FIG. 5 shows one embodiment of the present invention.

FIG. 6 shows one embodiment of the present invention.

FIGS. 7(a)-7(b) show the results of an experiment comparing the metal enhanced fluorescence of IRDye® 800CW or Alexa Fluor® 680 labeled streptavidin with silver metal colloidal aggregates over dye alone. FIG. 7(a) shows the fluorescence intensity scan of various dye-spotted colloid preparations (A-F) after irradiation with an excitation wavelength. FIG. 7(b) is a chart showing the relative fluorescence enhancement of various dye-spotted colloid preparations (A-F) over the dye spotted alone.

FIG. 8 shows a chart comparing the metal enhanced fluorescence of IRDye® 800CW labeled streptavidin on plain glass and silaniated glass with various colloidal preparations (A-G). The chart also compares the relative fluorescence enhancement of colloid preparations containing a salt and those with no salt.

FIG. 9(a)-9(b) show the results of an experiment demonstrating the metal enhanced fluorescence of IRDye® 800CW or Alexa Fluor® 680 labeled streptavidin with silver metal colloidal aggregates over dye alone on a nitrocellulose membrane substrate. FIG. 9(a) shows the fluorescence intensity scan of various dye-spotted colloid preparations after irradiation with an excitation wavelength. FIG. 9(b) is a chart showing the relative fluorescence enhancement of dye-spotted colloid preparations over the dye spotted alone.

FIG. 10(a) is a chart showing the metal enhanced fluorescence of IRDye® 800CW or IRDye® 700 labeled streptavidin on a silver island film coated glass substrate, known in the art, over dye alone. FIG. 10(b) is a chart showing the metal enhanced fluorescence of IRDye® 800CW or IRDye® 700 labeled streptavidin on a silver colloid coated glass substrate, known in the art, over dye alone.

FIG. 11 illustrates the results from an assay of a protein array with and without metal colloidal aggregates.

FIG. 12 is chart comparing an assay of the present invention to an assay without colloidal aggregates.

FIG. 13 shows an Image of an embodiment of a FLISA test reaction.

FIG. 14 shows an embodiment of a Limit of Detection experiment.

FIG. 15 shows a graph of one embodiment of integrated intensity verses concentration of IL-8.

### DETAILED DESCRIPTION OF THE INVENTION

### I. Definitions

As used herein, the terms “fluorescent dye,” “fluorescent group,” “fluorescent probe,” “fluorophore” and “fluorescent group” are used interchangeably and have equivalent meaning, and include a substance that emits electromagnetic energy such as light at a certain wavelength (the emission wavelength) when the substance is irradiated with radiation of a different wavelength (excitation wavelength). Exemplary fluorophores of the invention include those that are listed in the Molecular Probes Catalog (see, R. Haugland, The Handbook—A Guide to Fluorescent Probes and Labeling Technologies, 10^{th} Edition, Molecular probes, Inc. (2005)) which are incorporated herein by reference.

The term “chemiluminescent group” as used in this application includes a luminescent molecule, e.g., one capable of emitting light, that emits a characteristic frequency and corresponding wavelength of light as a result of the generation of electronically excited states formed as a result of a chemical reaction and therefore in response to a chemical agent capable of generating the electronically excited states by so reacting. A chemiluminescent group is similar to a fluorescent group, in the sense that light at a certain wavelength is emitted, but differs, in that the excited state of the molecule for a chemiluminescent group is generated by chemical transformation of the group rather than from photochemical excitation. Examples of chemiluminescent molecules include, but are not limited to, acridinium compounds, benzacridinium compounds, quinolinium compounds, isoquinolinium compounds, phenanthridinium compounds, luminol compounds, isoluminol compounds, and lucigenin compounds.

As used herein, the term “specific binding pair” includes two different molecules wherein one of the molecules has an affinity to bind to the second molecule through either non-covalent, or covalent means. Examples of specific binding pairs include, but are not limited to, antigen/antibody, secondary antibody/primary antibody, protein/carbohydrate, DNA/cDNA, RNA/cRNA, enzyme/substrate, receptor/ligand, among many others.

As used herein, the term “biomolecule” includes a natural or synthetic molecule for use in biological systems. Examples of biomolecules include, but are not limited to, antigens, steroids, vitamins, drugs, haptens, metabolites, toxins, environmental pollutants, amino acids, peptides, proteins, nucleotides, oligonucleotides, nucleic acids, (nuclei acid polymers) carbohydrates, lipids, and non-biological polymers. A biomolecule can also function as a first member of a specific binding pair.

As used herein, the term “metal colloidal aggregate,” or “colloidal aggregate” includes a colloidal aggregate comprising at least two metal colloids and an aggregating agent (e.g., a salt). A metal colloid can be of any geometry (e.g., spheroid, ellipsoid or a combination thereof). A metal colloidal aggregate can also be of any geometry (e.g., spheroid, ellipsoid, rod-like, etc.) and will typically have a diameter at its widest point on the surface of the substrate of at least 10 nm (e.g., 50 nm), or greater. Preferably, a metal colloidal aggregate has a diameter of at least 200 nm (e.g., 250 nm) to 3 μm, more preferably between about 500 nm (e.g., 750 nm) to 2 μm, and even more preferably between about 700 nm to 1.5 μm. Various metals include, for example, silver, gold, aluminum, rhenium, ruthenium, rhodium, palladium, silver, copper, osmium, iridium, platinum, or a combination thereof. Suitable aggregating agents include for example, alkali metal salts, alkaline metal salts, organic salts, transition metal salts, or a combination thereof.

As used herein, the term “aggregating agent” includes salts and other agents that promote aggregation by specific surface properties of unaggregated colloidal particles. In certain instances, a first lot of unaggregated colloidal particles is coated with a substance (e.g., 2-aminoethanethiol) to confer a positive charge on the particle surface. A second lot is separately coated to confer a negative charge (e.g., with 3-mercaptopropionic acid) on a particle surface. Aggregation occurs when the two lots are mixed, with positive-charged particles being attracted to negative-charged particles. Specific aggregation properties are controlled by the surface charge density built into one or both lots of colloids. In certain instances, particles are separately coated with two different, but complementary, binding agents prior to aggregation. In yet another embodiment, colloids are coated with an alkane thiol in an organic solvent to confer a hydrophobic surface on the particle surface. In this case, aggregation occurs by hydrophobic interaction when the colloids are diluted with an aqueous medium. Surface properties, in governing the binding affinity between coated aggregates and dye moieties, are useful in optimizing both metal-enhanced fluorescence and metal-enhanced optical absorbance of specific dye moieties.

As used herein, the term “excitation radiation” includes an amount of radiation that causes a molecule, typically a fluorophore, to emit radiation.

As used herein, the phrases “attaches to,” “attached to,” “binds to,” “associated with,” includes an association between two molecules (e.g., a binding group and a biomolecule), and includes both covalent and non-covalent (i.e., ionic, Van der Waals, and the like) interactions between the two molecules that occurs to maintain association with each other.

### II. Compositions

Metal enhanced fluorescence (MEF) is a well studied phenomenon wherein a fluorophore that is located in proximity to a metal surface exhibits enhanced (or increased) fluorescence emission upon irradiation with radiation at its excitation wavelength. The metal enhanced fluorescence effect is derived from the interaction of the dipole moment of the excited fluorophore with the surface of the metal resulting in an increase in the radiative decay rate, an increase in the absorption cross section, and a stronger (i.e., enhanced) fluorescence emission. The observed result, in practical terms, is that fluorophores (e.g., fluorophores with either low or high quantum yields) that are located near or proximate the metal surface, can be transformed into more efficient fluorophores. The present invention is predicated in-part on the surprising discovery that a surface that comprises at least one metal colloidal aggregate disposed thereon, is especially useful in metal-enhanced-fluorescence applications, and in particular, biological applications.

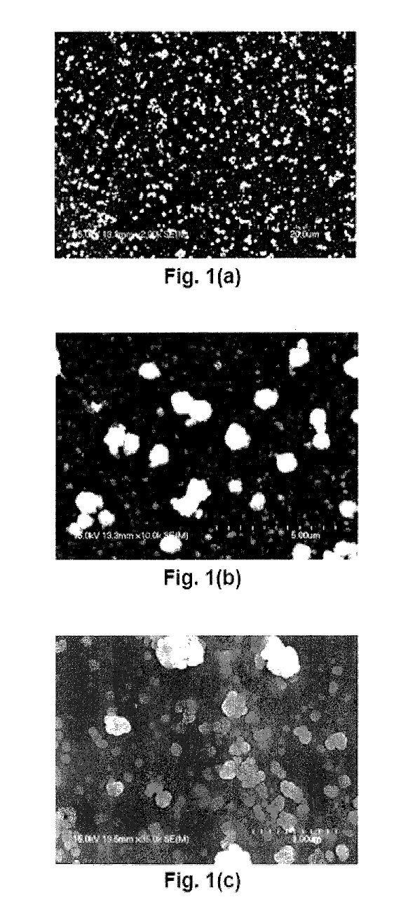

In one aspect, the present invention provides a composition that is a substrate having a metal colloidal aggregate disposed thereon, wherein a metal colloidal aggregate comprises at least two metal colloids and at least one salt. The metal colloids can be of any shape, e.g., spherical, ellipsoidal, and the like, and the resultant metal colloidal aggregate formed therefrom can also be of any three-dimensional shape, e.g., spherical, ellipsoidal, nanorod, nanotriangle, irregular, and the like. Preferably, a metal colloidal aggregate is an irregular amorphous shape, i.e., lacking symmetry. A colloidal aggregate of the present invention typically has a diameter at its widest point of between about 10 nm (e.g., 50 nm) to 3 μm, more preferably, about 50 nm to about 3 μm, more preferably about 100 nm (e.g., 250 nm) to about 3 μm, even more preferably, about 200 nm (e.g., 500 nm) to 3 μm, more preferably between 500 nm and 2 μm, and even more preferably, a diameter between about 750 nm and 1.5 μm. The diameter of a metal colloidal aggregate can be visualized and measured by techniques known in the art, e.g., scanning electron microscopy (SEM), dynamic light scattering, and the like. In certain instances, using SEM microscopy, a skilled artisan will be able to visualize the individual metal colloids combined together to form a metal colloidal aggregate of the present invention (see, e.g., FIGS. 1(a)-1(c)). A colloidal aggregate of the present invention is formed from the aggregation of at least two metal colloids. Preferably, a colloidal aggregate comprises at least 2-5 metal colloids. More preferably, a colloidal aggregate comprises at least 2-10 metal colloids, such as for example, 2, 3, 4, 5, 6, 7, 8, 9 or 10 metal colloids. In certain other instances, the number of aggregated colloids exceeds 10 such as 15, 20, 30, 40 or even more.

Suitable metals for use in a metal colloidal aggregate including, but not limited to, silver, gold, aluminum, rhenium, ruthenium, rhodium, palladium, silver, copper, osmium, iridium, platinum and a combination thereof. In one embodiment, the metal used in a colloidal aggregate of the invention includes, silver, gold, aluminum, or a mixture thereof. In another embodiment, the metal is silver or gold. In yet another embodiment, the metal is silver.

Salts that are useful for a metal colloidal aggregate of the invention include, for example, an alkali metal salt, an alkaline metal salt, an organic salt, a transition metal salt and a combination thereof. In one embodiment, the salt(s) in a metal colloidal aggregate are those that are present in biological buffer systems, such as for example, MES, Bis-Tris, PIPES (no salt), ACES, MOPS, TES, HEPES (no salt), HEPPS, Tricine, Bicine, CHES, CAPS MOPSO, DIPSO, HEPPSO, POPSO, AMPSO and CAPSO, Tris acetate-EDTA, Tris-borate-EDTA, Tris-Glycine, Tris-Tricine, Tris-Glycine-SDS, Tris-Tricine-SDS, Tris, Phosphate Buffered Saline (PBS), Sodium Chloride-Sodium Citrate and EDTA buffers, and the like (see, Ferguson W. J., et al., Anal. Biochem. 104: 300 (1980); Good, N. E., (1966) Biochemistry, 5, 467-477); and as described in Protein Purification: Principles and Practice 3rd ed. Robert K. Scopes, Springer (1994). A skilled artisan would recognize additional buffers systems or minor variations of the above buffer systems that would also comprise salts that are also useful in a colloidal aggregate of the present invention. An inventive metal colloidal aggregate comprises at least one type of salt, more preferably, a colloidal aggregate comprises a mixture of salts.

In one embodiment, a metal colloidal aggregate comprises at least one salt selected from the group of NaCl, KCl, Na₂HPO₄, NaH₂PO₄, KH₂PO₄, LiCl, Tris(hydroxymethyl)aminomethane hydrochloride (Tris-HCl), CaCl₂, NH₄OAc, MgCl₂, KOAc, NaOAc, succinic acid (disodium salt), Tris-acetate, Citric acid (Trisodium), NaOH, KOH, and a combination thereof. In another embodiment, the at least one salt is selected from the group consisting of NaCl, KCl, Na₂HPO₄, NaH₂PO₄, KH₂PO₄, LiCl and Tris(hydroxymethyl)aminomethane hydrochloride (Tris-HCl), and a combination thereof.

The weight ratio, or stoichiometry of metal:salt in a colloidal aggregate can be from 99.999:0.001 to 50:50. Preferably, the ratio of metal:salt in a colloidal aggregate is from about 99.9:0.1 to about 50:50, or from about 99.9:0.1 to about 75:25, or from about 99.9:0.1 to about 85:15, or from about 99.9:0.1 to about 90:10, or from about 99.9:0.1 to about 95:5, even more preferably, from about 99.9:0.1 to about 98:2.

In another embodiment, a metal colloidal aggregate of the invention is proximate (e.g., disposed) on a substrate (e.g., surface). The substrates can be metallic or non-metallic. Suitable substrates of the invention are generally those that are compatible for use in biological assays. These substrates include for example, glass, silanized glass, a quartz plate, plastic, a carbon nanotube, a metal and a membrane. Membranes suitable for use as a substrate composition include nylon, polyvinylidine difluoride (PVDF), nitrocellulose, and the like. Plastics suitable for the compositions include, but are not limited to, polypropylene, polystyrene, polycarbonate, and the like. In one embodiment, the substrate is selected from the group consisting of glass, silanized glass, a quartz plate, plastic, a carbon nanotube, a metal and a membrane. In another embodiment, the substrate is selected from the group consisting of glass, silanized glass, a nylon membrane, polyvinylidine difluoride (PVDF) membrane, a nitrocellulose membrane, polypropylene, polystyrene, polyvinylchloride and polycarbonate. In yet another embodiment, the substrate is parylene.

Other materials suitable for use the present invention as a substrate include silicon, silica, quartz, glass, controlled pore glass, carbon, alumina, titania, tantalum oxide, germanium, silicon nitride, zeolites, and gallium arsenide. Many metals such as gold, platinum, aluminum, copper, titanium, and their alloys are also options for substrates of the array. In addition, many ceramics and polymers may also be used as substrates. Polymers which may be used as substrates include, but are not limited to, the following: polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyalkenesulfone (PAS); polypropylethylene, polyethylene; polyhydroxyethylmethacrylate (HEMA); polydimethylsiloxane; polyacrylamide; polyimide; and block-copolymers. Preferred substrates include silicon, silica, glass, and polymers.

In certain instances, the substrate may optionally further comprise a coating. This coating may either be formed on the substrate or applied to the substrate. The substrate can be modified with a coating by using thin-film technology based, for instance, on physical vapor deposition (PVD), plasma-enhanced chemical vapor deposition (PECVD), or thermal processing. Alternatively, plasma exposure can be used to directly activate or alter the substrate and create a coating. For instance, plasma etch procedures can be used to oxidize a polymeric surface (for example, polystyrene or polyethylene to expose polar functionalities such as hydroxyls, carboxylic acids, aldehydes and the like) which then acts as a coating.

Additionally, the substrates alone, or with a metal colloidal aggregate already disposed thereon can be coated with a polymer, a gel, an adhesive, an oxide, SiO₂, a mercaptan, bovine serum albumin (BSA), casein, DNA or other biological materials including non-mammalian protein mixtures. A coating layer added to a colloidal aggregate substrate compositions is beneficial for a variety of reason, including, for example, i) for protection of a colloidal aggregate to prevent its degradation; ii) as a spacer to maintain the distance between a colloidal metal aggregate and the fluorophore to achieve optimal fluorescence enhancement; iii) to coat and make non-reactive (inert) the substrate material itself; and iv) to increase the affinity or “adhesiveness” of the substrate for other molecules (e.g., human serum albumin coating on the substrate increases its affinity for binding to colloids; and silver and gold colloids bind to glass or polymer surfaces coated with functional groups such as CN, NH₂, or SH with high affinity (Freeman R. G., et al., Science, 267, 1629-1632)). For example, a SiO₂ coating on a colloidal aggregate substrate composition can increase the stability of metals that are otherwise prone to oxidation, such as silver. In one embodiment, a colloidal aggregate is coated with SiO₂. A coating layer of SiO₂ in a thickness that does not affect the metal enhanced fluorescence capability of a metal colloidal aggregate is preferred. Typically, a coating layer SiO₂ of about 5 nm to about 30 nm, or between about 10 nm to about 15 nm is preferred.

The inventive substrates having a metal colloidal aggregate are especially useful in biological applications, e.g., in fluorescence-based assays, in which metal enhanced fluorescence is beneficial for the advantageous enhanced detection of a fluorophore.

In preferred aspects, a binding member is immobilized on the substrate either directly, through a linker, via a coating, via a metal colloidal aggregate and combinations thereof. Those of skill in the art know of various methods of attaching binding members to substrates (see, for example, U.S. Pat. No. 6,329,209, incorporated herein by reference).

In yet another aspect, the present invention provides for a system for detection of a specific binding pair, the system comprising: a substrate having at least one metal colloidal aggregate disposed thereon; optionally a biomolecule located proximate to the colloidal aggregate; and a first member of a specific binding pair located proximate to the colloidal aggregate, or attached to the biomolecule, if present. In another embodiment, the system comprises a second member of the specific binding pair.

Suitable binding pairs that can be detected using the inventive system include, but are not limited to, protein/antibody, DNA/cDNA, RNA/cRNA, antigen/antibody, secondary antibody/antibody, peptide nucleic acid/complementary strand, protein/carbohydrate, drug/drug receptor, toxin/toxin receptor, carbohydrate/lectin or carbohydrate receptor, peptide/peptide receptor, protein/protein receptor, peptide/small molecule, nucleic acid/protein, nucleic acid/enzyme, receptor/small molecule, enzyme/substrate, DNAzyme/substrate, RNAzyme/substrate, protein/protein, aptmer/ligand, hormone/hormone receptor, ion/chelator, biotin/avidin, biotin/streptavidin, biotin/neutravidin, folate/folate-binding protein and IgG/protein A or protein G, phosphorylated and non-phosphorylated binding pairs.

In one aspect of the present invention, one member of the specific binding pair will have attached thereto an enzyme, preferably, e.g., a hydrolytic enzyme attached thereto. Preferably, the hydrolytic enzyme is selected from phosphatases, glycosidases, peptidases, proteases, and esterases. In one embodiment, the second member of the specific binding pair will have an enzyme attached thereto.

In certain aspects, at least one member of the specific binding pair (e.g., first member, second member, both members, and the like) will have attached thereto an enzyme which activates a detectable group. In one embodiment, the detectable group is a chemiluminescent group. Chemiluminescent groups contemplated for the invention include, but are not limited to, acridinium compounds, benzacridinium compounds, quinolinium compounds, isoquinolinium compounds, phenanthridinium compounds, luminol compounds, isoluminol compounds, and lucigenin compounds.

In another embodiment, the detectable group is a fluorogenic group (e.g., a fluorescent dye). Exemplary fluorophores suitable for use in the present invention include those listed in the Molecular Probes Catalogue, which is herein incorporated by reference (see, R. Haugland, The Handbook—A Guide to Fluorescent Probes and Labeling Technologies, 10^{th} Edition, Molecular probes, Inc. (2005)). Such exemplary fluorophores include, but are not limited to, Alexa Fluor® 350, Dansyl Chloride (DNS-Cl), 5-(iodoacetamida)fluoroscein (5-IAF); fluoroscein 5-isothiocyanate (FITC), tetramethylrhodamine 5-(and 6-)isothiocyanate (TRITC), 6-acryloyl-2-dimethylaminonaphthalene (acrylodan), 7-nitrobenzo-2-oxa-1,3,-diazol-4-yl chloride (NBD-Cl), ethidium bromide, Lucifer Yellow, 5-carboxyrhodamine 6G hydrochloride, Lissamine rhodamine B sulfonyl chloride, Texas Red™ sulfonyl chloride, BODIPY™, naphthalamine sulfonic acids, including, but not limited to, 1-anilinonaphthalene-8-sulfonic acid (ANS) and 6-(p-toluidinyl)naphthalen-e-2-sulfonic acid (TNS), Anthroyl fatty acid, DPH, Parinaric acid, TMA-DPH, Fluorenyl fatty acid, Fluorescein-phosphatidylethanolamine, Texas red-phosphatidylethanolamine, Pyrenyl-phophatidylcholine, Fluorenyl-phosphotidylcholine, Merocyanine 540, 1-(3-sulfonatopropyl)-4-[β-[2[(di-n-butylamino)-6naphthyl]vinyl]pyridinium betaine (Naphtyl Styryl), 3,3′dipropylthiadicarbocyanine(diS—C₃-(5)), 4-(p-dipentyl aminostyryl)-1-methylpyridinium (di-5-ASP), Cy-3 Iodo Acetamide, Cy-5-N-Hydroxysuccinimide, Cy-7-Isothiocyanate, rhodamine 800, IR-125, Thiazole Orange, Azure B, Nile Blue, Al Phthalocyanine, Oxaxine 1,4′,6-diamidino-2-phenylindole (DAPI), Hoechst 33342, TOTO, Acridine Orange, Ethidium Homodimer, N(ethoxycarbonylmethyl)-6-methoxyquinolinium (MQAE), Fura-2, Calcium Green, Carboxy SNARF-6, BAPTA, coumarin, phytofluors, Coronene, metal-ligand complexes, IRDye® 700DX, IRDye® 700, IRDye® 800RS, IRDye® 800CW, IRDye® 800, Alexa Fluor® 680, Alexa Fluor® 700, Alexa Fluor® 750, Alexa Fluor® 790, Cy5, Cy5.5, Cy7, DY 676, DY680, DY682 and DY780. Additional suitable fluorophores include enzyme-cofactors; lanthanide, green fluorescent protein, yellow fluorescent protein, red fluorescent protein, or mutants and derivates thereof. In one embodiment of the invention, the second member of the specific binding pair has a detectable group attached thereto.

Typically, the fluorescent group is a fluorophore selected from the category of dyes comprising polymethines, pthalocyanines, cyanines, xanthenes, fluorenes, rhodamines, coumarins, fluoresceins and BODIPY™.

In one embodiment, the fluorescent group is a near-infrared (NIR) fluorophore that emits in the range of between about 650 to about 900 nm. Use of near infrared fluorescence technology is advantageous in biological assays as it substantially eliminates or reduces background from auto fluorescence of biosubstrates. Another benefit to the near-IR fluorescent technology is that the scattered light from the excitation source is greatly reduced since the scattering intensity is proportional to the inverse fourth power of the wavelength. Low background fluorescence and low scattering result in a high signal to noise ratio, which is essential for highly sensitive detection. Furthermore, the optically transparent window in the near-IR region (650 nm to 900 nm) in biological tissue makes NIR fluorescence a valuable technology for in vivo imaging and subcellular detection applications that require the transmission of light through biological components. Within aspects of this embodiment, the fluorescent group is preferably selected form the group consisting of IRDye® 700DX, IRDye® 700, IRDye® 800RS, IRDye® 800CW, IRDye® 800, Alexa Fluor® 680, Alexa Fluor® 700, Alexa Fluor® 750, Alexa Fluor® 790, Cy5, Cy5.5, Cy7, DY 676, DY680, DY682 and DY780. In certain embodiments, the near infrared group is IRDye® 800CW, IRDye® 800, IRDye® 700DX, IRDye® 700, Dynomic DY676 or Alexa Fluor® 680.

Optionally, in certain aspects, one member of a specific binding pair has attached thereto, an acceptor fluorophore or a quencher. The fluorescent (donor) group and the quencher or acceptor fluorophore can be located on the same member of the specific binding pair, typically on the second member of the binding pair; alternatively, the fluorescent group (e.g., donor) is located on one member of a specific binding pair and the acceptor fluorophore or quencher is located on the complementary member of the specific binding pair.

In certain other aspects, the binding of a first member of a specific binding pair with a second member of a specific binding pair will bring the fluorescent group, or the fluorescent (donor) group/quencher, or acceptor fluorophore pair to within a sufficient distance from a metal colloidal aggregate to result in the enhancement of fluorescence emission upon irradiation of the fluorescent group. The term “sufficient distance” includes a distance between the fluorescent group and a metal colloidal aggregate that results in the enhancement of fluorescence emission upon irradiation with excitation radiation of the fluorescent group. Typically, this distance is from about 25 Å to about 1000 Å, more preferably about 50 Å to about 200 Å.

In certain other instances, the dyes useful herein are disclosed in U.S. Patent Publication No. US2007/0042398, to Peng et al. published Feb. 22, 2007, and incorporated herein by reference in its entirety. As discussed therein, the novel near IR quenching cyanine dyes have absorption wavelengths in the near-infrared region of about 650-900 nm and are essentially non-fluorescent. In this embodiment, binding is detected by fluorescence quenching.

In one embodiment, the system for detecting a specific binding pair comprises: a substrate having at least one colloidal aggregate disposed thereon; optionally a biomolecule located on or proximate to the colloidal aggregate; a first member of a specific binding pair located proximate to the colloidal aggregate, or attached to the biomolecule, if present; and further comprising the second member of a specific binding pair, wherein the second member of a specific binding pair has at least one detectable group, such as a fluorescent group, attached thereto. Within one aspect, a second member of the binding pair comprises a plurality of fluorescent groups, i.e., at least two fluorophores. Within another aspect of this embodiment, a second member of the binding pair further comprises a quencher or acceptor fluorophore. The binding of a first member of the specific binding pair to a second member having a fluorescent group attached thereto, brings the at least one fluorescent group to a sufficient distance from a metal colloidal aggregate, i.e., within the fluorescence enhancement region, to result in enhancement of fluorescence emission upon irradiation. In certain aspects, a sufficient distance is between about 10 Å to about 2000 Å, preferably between about 25 Å to about 1000 Å, more preferably, between about 50 Å to about 200 Å.

### III. Preparation of a Metal Colloidal Aggregate Substrate

The preparation of metal colloids is generally known in the art. Conventionally, substrates comprising metal colloids deposited thereon can be prepared by citrate reduction of the metals. The size of metal colloids prepared by standard methods (e.g., citrate reduction) are generally small, generally in the range of about 4 nm to about 120 nm.

In contrast, the inventive substrate composition comprises metal colloidal aggregates that are larger and comprise a salt, or other aggregation generating substance and are produced by the controlled aggregation of metal colloids. The inventive substrate compositions are prepared by a controlled aggregation process. The steps in the controlled aggregation process include, for example: a) concentrating a metal colloid suspension by, for example, centrifugation; b) next, a salt solution, typically a buffer solution, is then added to the concentrated metal colloids to produce controlled aggregation of the colloids, which is then; c) applied onto a substrate surface; and d) optionally dried.

In one embodiment, in step b), the final concentration of salt in a colloidal solution can be from about 0.05 mM to about 200 mM. Preferably, the final concentration of salt in a colloidal solution is from about 0.5 mM to about 100 mM, even more preferably, from about 1 mM to about 50 mM. In one embodiment, a metal colloidal aggregate substrate composition is prepared using a 0.1×PBS or 15 mM Tris pH 8 salt solution. Within certain aspects of this embodiment, the 0.1×PBS or 15 mM Tris pH 8 salt solution is added to the concentrated colloidal suspension of step a) in a equal volume mixture, to result in a colloidal aggregate solution of step b) that has a concentration of 0.05×PBS or 7.5 mM Tris pH 8 salt solution.

Without being bound by any particular theory, it is believed that the formation of a metal colloidal aggregate is due to the shielding charges effect of the added salts on the metal colloids. In more detail, in the formation of silver colloids, the sodium citrate acts as both a reducing and a stabilizing agent in the chemical reduction of silver salts (e.g., silver nitrate). The negative citrate ions are associated with the silver colloids via an electrostatic interaction which stabilizes the colloidal suspension. The repulsion of like charges of the citrate ions prevents aggregation of the colloids. Typically, sodium hydroxide is added as a pH modifying agent and is a factor in controlling the size of the individual colloids. To form a colloidal aggregate of the present invention, a dilute salt solution is added which provides counterions that can partially shield the electrostatic interactions between the colloids in suspension. Upon reduction of the electrostatic repulsion between individual colloids, van der Waals forces tend to cluster the colloids, to form an inventive colloidal aggregate.

In some embodiments, the controlled aggregation process produces a monodisperse population of metal colloidal aggregates. In some other embodiments, the controlled aggregation process described above produces a polydisperse population of metal colloidal aggregates. In certain embodiments, the substrate composition comprises at least a 99% monodisperse population of colloidal aggregates. In other embodiments, a colloidal aggregate on the substrate composition is at least a 90% monodisperse population, or at least an 80% monodisperse population, or at least a 70% monodisperse population, or at least a 60% monodisperse population, or at least a 50% monodisperse population, or least a 40% monodisperse population, or at least a 30% monodisperse population, or at least a 20% monodisperse population, or at least a 10% monodisperse population, or at least a 5% monodisperse population. In certain other instances, the population of colloidal aggregates is a bell curve of various numbers of colloids making up the colloidal aggregates.

Although the following description uses “salts” as the aggregating agents, it is not intended to be limiting. Those of skill in the art will appreciate and know of other suitable aggregating agents that can be used. The concentration of the salt solution is one factor for controlling the aggregation of the colloids. If the salt concentration is too high, the colloids will aggregate into a single metal ball, which will provide little or no enhancement on a substrate surface. Typically, the salts that are present in the buffer solution are incorporated into a metal colloidal aggregate substrate composition. However, the salt selection for a colloidal aggregate is not limited to only those found in buffers. Additional salts that can impart desirable properties to the colloidal aggregate can also be separately added.

In an alternative embodiment, when a concentrated colloid solution in which “no salt” has been added is deposited onto a substrate and dried, this “no-salt” substrate preparation can be spotted with a salt solution at a later time and re-dried to form a metal colloidal aggregate substrate of the invention having enhanced fluorescence.

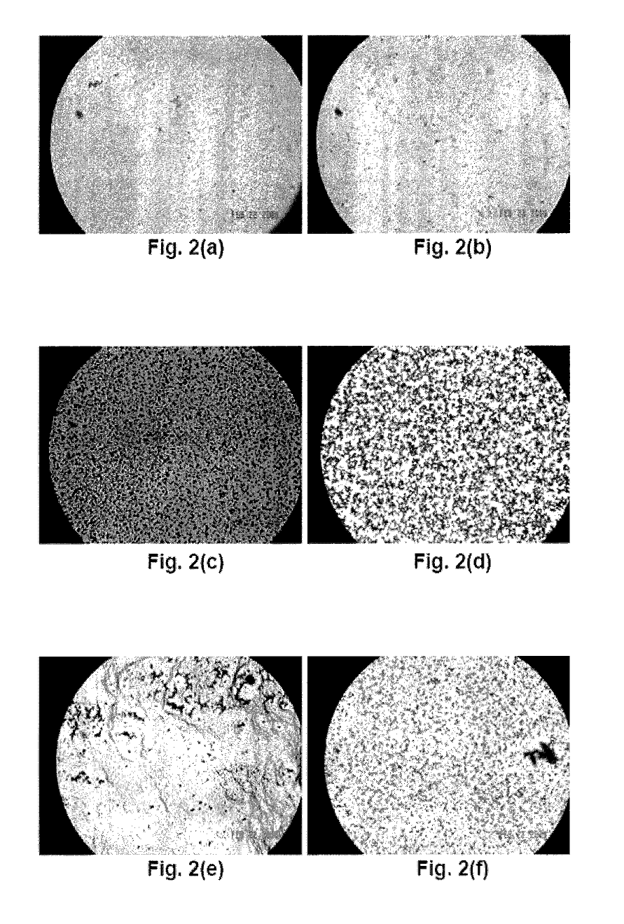

The morphology of a colloidal aggregate coated surface of the substrate is visibly altered when using a controlled aggregation processes of the present invention. For example, scanning electron microscopy images of an inventive silver colloidal aggregate substrate composition show that the aggregated colloidal particles are large, e.g. having a diameter of roughly about 750 to about 1.5 μm (FIG. 1(a)-1(c)). The difference between the metal surfaces of the inventive substrate compositions as compared to those known in the art can be visually assessed in FIGS. 2(a)-2(f). These images depict for example, microscope images of an inventive metal colloidal aggregate prepared by controlled aggregation of colloids on various surfaces, i.e., plain glass (FIGS. 2(c), 2(f)), silaniated glass (FIG. 2(d)), a regular colloid coated substrate on plain glass (FIG. 2(b)), and a silver island film on plain glass (FIG. 2(a)). The images in FIGS. 2(c), 2(d) and 2(f) of colloidal aggregates of the invention on glass substrates clearly show that the inventive aggregates are larger and distributed more evenly than those of the prior art (FIGS. 2(a) and 2(b)) or those prepared as by a non-controlled aggregation process (FIG. 2(e)). A colloidal aggregate can be disposed onto a substrate surface in a random pattern, i.e., no pattern; or alternatively, a colloidal aggregate can be disposed onto the substrate surface as a periodic array of metal colloidal aggregates using, for example, nanosphere lithography techniques to control the size, shape and spacing of an inventive metal colloidal aggregate.

The size of a colloid and their composition can be determined by the extensive publications on the optical properties of metal particles and the effects of interface chemistry on the optical property of colloids. (see, Krelbig, U., Gartz, M., and Hilger, A., Mie resonances: Sensors for physical and chemical cluster interface properties, Ber. Bunsenges, Phys. Chem., 101(11). 1593-1604 (1997)).

In certain embodiments, the aggregates (both pre- and post formation) are coated such as to produce a stable near-IR enhancement reagent to improve reproducibility and limit of detection (LOD) values. Advantageously, in certain instances, the coatings stabilize the colloidal aggregates and also provide for example, a suitable spacer optimizing the distance between fluorophores and the metal. This can enhance MEF activity. Suitable coatings include, but are not limited to, metal-binding compounds, including 1) protein-based blocking buffers; 2) mercaptans such as dithiotheritol (DTT) (Nogueria, H. I. S. et al., Journal of Materials Chemistry 12:2339-2342 (2002); Graf, C., et al., Langmuir 19:6693-6700 (2003)); 3) PEG-conjugated 3,4-dihydroxy-L-phenylalanine (DOPA)₃ (Dalsin, J. L. et al., Langmuir 21:640-646 (2005)) (Nerites, Corp.); 4) silica films (Graf, C. et al., Langmuir 19:6693-6700 (2003); Ung, T. et al., Langmuir 14:3740-3748 (1998); Wong, C. et al., Journal of Young Investigators 6(1) (2002); Stober, W. et al., J Colloid Interface Sci 26:62-69 (1968)), or 5) underivatized polyethylene glycol (PEG) of various lengths (Murcia, M. J. et al., in Nanotechnologies for the Life Sciences C. S. S. R. Kumar, Editor. WILEY-VCH. p. 40 (2005)).

In one aspect, a blocking buffer (e.g., commerically available from LI-COR Cat. #927-40000) and DTT are used. The colloidal aggregates can be suspended in a solution of each tested material, then washed by centrifugation to remove excess coating material and normalized in concentration on the basis of light-scattering activity.

### IV. Method of Use

The inventive systems and methods for detecting the presence of a specific binding pair are especially useful in biological applications, e.g., fluorescence or chemiluminescence assays, and the like, wherein metal enhanced detection of a detectable group, such as a fluorescent group, is desired. In one aspect, the fluorescent group has been liberated by a protease from the binding member to increase the Limit of Detection (LOD).

In certain aspects, the present invention provides a method for detecting the presence of a specific binding pair in a sample. In one embodiment, the method (Method A) comprises:
i) providing a substrate having at least one biomolecule disposed thereon and at least one metal colloidal aggregate proximate thereto;
ii) contacting the substrate with a first member of a specific binding pair wherein the first member of the specific binding pair attaches to the biomolecule;
iii) contacting the substrate with a second member of the specific binding pair; wherein the second member of a specific binding pair comprises at least one fluorescent group attached thereto; and
iv) irradiating the at least one fluorescent group with excitation radiation to detect the fluorescent group.

In biological applications, the detection of the specific binding pair is useful to identify the binding pair itself (e.g., in DNA hybridization assays), or to indirectly identify the presence of a different biomolecule of interest in an assay system through the binding of that biomolecule to a member of a specific binding pair (e.g., western blot). Additionally, it should be readily apparent to the skilled artisan that when the inventive system is used to indirectly identify the presence of a different biomolecule of interest in an assay system, that the biomolecule need not be directly attached to the specific binding pair that is detected. Instead, there can be numerous specific binding pairs intervening the attachment of the biomolecule of the interest with the specific binding pair that is ultimately detected; in other words, the biomolecule and the specific binding pair that is ultimately detected can be linked, by covalent or non-covalent means, through a series of specific binding pairs.

In a variation of Method A, the biomolecule in step i) is the first member of a specific binding pair, and in this embodiment, step ii) of method A is eliminated. In this embodiment, the method (Method A1) comprises:
i) providing a substrate having a first member of a specific binding pair disposed thereon and at least one metal colloidal aggregate proximate thereto;
ii) contacting the substrate with a second member of the specific binding pair; wherein the second member of a specific binding pair comprises at least one fluorescent group attached thereto; and
iii) irradiating the at least one fluorescent group with excitation radiation to detect the fluorescent group.

In another variation of Method A, in step iii) the fluorescent group is replaced with a enzyme, preferably a hydrolytic enzyme. In this method (Method B), in step iv) a chemiluminescent compound is added to the enzyme, which converts the compound to a luminescent product which is detected. Specifically, Method B comprises
i) providing a substrate having at least one biomolecule disposed thereon and at least one metal colloidal aggregate proximate thereto;
ii) contacting the substrate with a first member of a specific binding pair wherein the first member of the specific binding pair attaches to the biomolecule;
iii) contacting the substrate with a sample containing a second member of the specific binding pair; wherein the second member of a specific binding pair comprises at least one enzyme group attached thereto;
iv) incubating the substrate with a chemiluminescent compound to detect a luminescent product.

Similarly, in a variation of Method B, the biomolecule in step i) is the first member of a specific binding pair, and in this embodiment, step ii) of method B is eliminated. In this embodiment, the method (Method B1) comprises:
i) providing a substrate having a first member of a specific binding pair disposed thereon and at least one metal colloidal aggregate proximate thereto;
ii) contacting the substrate with a second member of the specific binding pair; wherein the second member of a specific binding pair comprises at least one enzyme group attached thereto;
iii) incubating the substrate with a chemiluminescent compound to detect a luminescent product.

Preferably, the hydrolytic enzymes used in Method B or B1 include phosphatases, glycosidases, peptidases, proteases, and esterases. By far, most commonly used enzymes are phosphatases and glycosidases. Preferably, the chemiluminescent compound is selected from the group of acridinium compounds, benzacridinium compounds, quinolinium compounds, isoquinolinium compounds, phenanthridinium compounds, luminol compounds, isoluminol compounds, and lucigenin compounds.

In one embodiment of either Method A or A1, the second member of the specific binding pair further comprises an acceptor fluorophore or quencher group. In another embodiment of either Method A or A1, the second member of the specific binding pair comprises a plurality of fluorophores attached thereto.

In still yet another embodiment of either Methods A or A1, the fluorescent group attached to the second member of a specific binding pair is a near-infrared fluorophore.

The binding of a first member of the specific binding pair to the second member of a binding pair having a fluorescent group attached thereto will bring the fluorescent group (or donor group) within a sufficient distance from a metal colloidal aggregate to result in the enhancement of fluorescence emission of the fluorophore upon irradiation with excitation radiation as set forth in step iii) of either method A or A1. In the presence of a metal colloidal aggregate, the enhancement of fluorescence emission can be greater than 150-fold as compared with the emission of the same fluorescent group that is irradiated in the absence of a metal colloidal aggregate.

The inventive substrates comprising a metal colloidal aggregate are superior at enhancing fluorescence of a fluorescent group than other types of metal surfaces, such as metal island films or standard metal colloid coated surfaces (see, Example 1). In one embodiment, as shown in Example 1, an inventive silver metal colloidal aggregate (i.e., disposed on a glass substrate enhances the fluorescence emission of IRDye® 800CW-labeled streptavidin is nearly 250-fold over the fluorescence emission of the dye alone. By comparison, traditional silver island films and colloid coated surfaces with IRDye® 800CW gave enhancements of only 6 to 16-fold over dye alone. Similarly, silver colloidal aggregates coated on nitrocellulose membrane (see, Example 3) enhanced the fluorescence emission of IRDye® 800CW and Alexa Fluor® 680 labeled streptavidin by nearly 20-fold. This 20-fold increase is clearly superior to traditional silver island films and colloid coated nitrocellulose membranes that are completely ineffective at enhancing fluorescence emission of the dye.

In certain aspects, the magnitude of enhancement is increased by liberating the fluorescent group from the binding member. One mechanism of liberation is by incubating with a protease to effectuate proteolytic digestion. Suitable proteases include, but are not limited to, HCV protease, HIV protease, CMV protease, secretase, capase, ADAM protease, matrix metalloprotease, cathepsin, bromolain, chymotrypsin, collagenase, elastase, kallikrein, papain, pepsin, plasmin, proteinase K, renin, streptokinase, substilisin, thermolysin, thrombin, trypsin, urokinase, and the like. Trypsin and proteinase K are especially preferred.

In another embodiment, the fluorescent group is released or liberated by for example, a chemical agent that disrupts non-covalent bonds between proteins and dyes. One such class of agents are detergents. Another class of agents are pH-altering agents (e.g., acids and bases). In yet another embodiment, the fluorescent group is released or liberated by chemically cleaving covalent bonds, for example, by reducing agents cleaving disulfide linkers joining dyes to antibodies. In still another embodiment, photocleavable bonds in dye-linkers are broken by radiation energy. In still yet a further embodiment, enzymatic cleavage is accomplished by cleaving carbohydrate linkers with a glucosylase, phosphatases, or by cleaving nucleic acid chains with nucleases.

While the magnitude of enhancement of fluorescence emission varies depending on the substrate used (in one aspect, a “hard” substrate such as glass typically provides greater enhancement than “soft” substrates, such as membranes), overall fluorescence enhancement of a fluorescent group provided by a metal colloidal aggregate of the invention is superior over the enhancement of the same fluorophore using standard metal island films or colloid coated substrates known in the art.

In one embodiment, the enhancement of fluorescence emission on a “hard” substrate, such as glass, quartz, is between about 5 to about 250-fold greater as compared to the emission of the same fluorescent group that is irradiated in the absence (i.e., not within the “sufficient distance”) of a metal colloidal aggregate. In certain aspects of this embodiment, the enhancement of fluorescence emission is between about 20 to about 250-fold greater. In yet another embodiment, the enhancement of fluorescence is between about 35 to about 250-fold greater. In another aspect of this embodiment, the enhancement of fluorescence is between about 50 to about 250-fold greater. In yet another embodiment, the enhancement of fluorescence emission on a “soft” substrate, such as a membrane, is between about 20 to about 60-fold greater. In certain aspects of this embodiment, the enhancement of fluorescence is about 5 to about 60-fold greater. In another aspect of this embodiment, the enhancement of fluorescence is about 10 to about 30-fold greater.

In certain aspects, the metal colloidal aggregates of the present invention can be used in various immunoassays, which include, but are not limited to, radioimmunoassay, enzyme-linked immunosorbent assay (ELISA), “sandwich” assays, precipitin reactions, gel diffusion immunodiffusion assay, agglutination assay, fluorescent immunoassays, protein A or G immunoassays and immunoelectrophoresis assays.

Monoclonal or polyclonal antibodies produced against antigens are useful in an immunoassay on samples of blood or blood products such as serum, plasma or the like, spinal fluid or other body fluid, e.g. saliva, urine, lymph, and the like, to diagnose patients with the characteristic disease state linked to the antigen. The antibodies can be used in any type of immunoassay. This includes both the two-site sandwich assay and the single site immunoassay of the non-competitive type, as well as in traditional competitive binding assays.

For ease and simplicity of detection, and its quantitative nature, the sandwich or double antibody assay of which a number of variations exist, are all contemplated by the present invention. For example, in a typical sandwich assay, unlabelled antibody is immobilized on a solid phase such as microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which be quantitated by comparison with control samples containing known amounts of antigen.

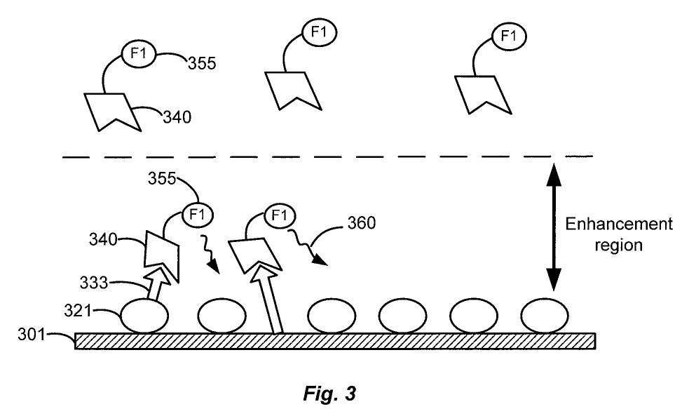

Various embodiments of the inventive methods for detecting specific binding pairs in a sample are illustrated in FIGS. 3-6 and described below.

In one embodiment (see, FIG. 3), the substrate 301 comprises a plurality of metal colloidal aggregates 321 disposed thereon. The first member of the specific binding pair 333 is attached to or located proximate to a metal colloidal aggregate 321. Alternatively, the first member of the specific binding pair 333 is disposed on a substrate 301 and a plurality of metal colloidal aggregates 321 are located proximate thereto. The complementary member of the binding pair 340, having an fluorescent group 355 attached thereto is added to the sample. Upon binding to the first member of the specific binding pair, the fluorescent group 355 is brought within a sufficient distance to a metal colloidal aggregate such that upon irradiation with excitation radiation, fluorescence emission (arrow 360) is enhanced.

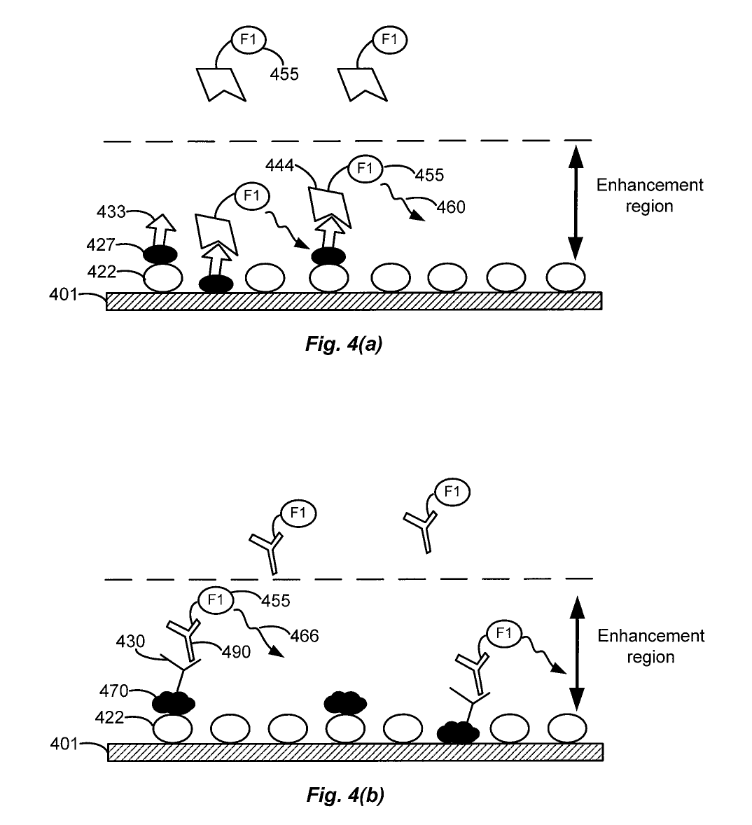

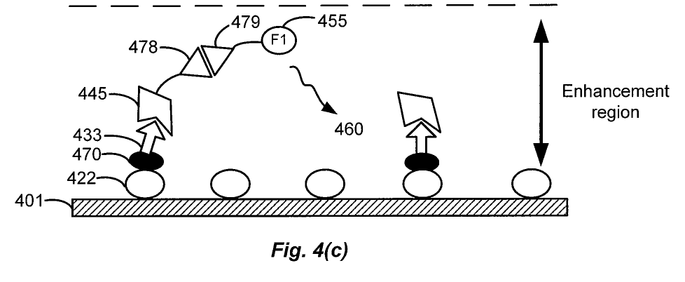

In another embodiment (see, FIG. 4A), the substrate 401 comprises a plurality of metal colloidal aggregates 422 disposed thereon and further comprises a biomolecule 427 located on, or proximate to a metal colloidal aggregate 422. Alternatively, the substrate 401 has a biomolecule 427 disposed thereon and a plurality of metal colloidal aggregates 422 are located proximate thereto. The first member of the specific binding pair 433 is added to the sample and binds to the biomolecule 427. Next, the complementary member of the binding pair 444, having an fluorescent group 455 attached thereto is added to the sample. Upon binding to the first member of the specific binding pair, the fluorescent group 455 is brought within a sufficient distance to a metal colloidal aggregate such that upon irradiation with excitation radiation, fluorescence emission (arrow 460) is enhanced.

A specific embodiment of the method outlined in FIG. 4(a), is shown in FIG. 4(b), which depicts a schematic of a model western blot assay. Separated protein biomolecules 470 from gel electrophoresis are transferred onto a substrate 401 (e.g., nitrocellulose or PVDF membrane) that later has a plurality of metal colloidal aggregates 422 disposed thereon. Alternatively, a plurality of metal colloidal aggregates 422 are located proximate thereto. The protein biomolecules 470 are located on the substrate, and/or proximate to a metal colloidal aggregate. To the sample is added the first member of a specific binding pair, i.e., a primary antibody 430, which binds to the protein biomolecules 470. Next, a secondary antibody 490 having a fluorescent group 455 attached thereto is added to the sample; and upon binding with the primary antibody 430, the fluorescent group 455 is brought within sufficient distance to a metal colloidal aggregate 422 that is added to the substrate such that upon irradiation with excitation radiation, enhanced emission (see arrow 466) of the fluorescence group is observed.

FIG. 4(c) illustrates yet another variation of the method outlined in FIG. 4(a). In a variation of a western blot assay using the inventive method as shown in FIG. 4(c), the biomolecule 470 (e.g., a protein) to be identified is deposited on a substrate 401 proximate to a colloidal metal aggregate 422 and is attached to a first member of a specific binding pair (1) 433 (e.g., a primary antibody). Alternatively, the colloidal metal aggregate 422 is disposed on the substrate, and the biomolecule is located on, or near the colloidal metal aggregate 422. The second member of the specific binding pair (1) 445 (e.g., a secondary antibody) having a first member of a specific binding group (2) 478 attached thereto (e.g., biotin group) is added to the system and binds to the first member 433. A second member of a specific binding pair (2) 479 (e.g., a fluorescent dye-labeled streptavidin) can then be added to bind to the biotin group 478. The binding of the specific binding pair of the fluorescent dye-labeled streptavidin and biotin is the binding pair that is ultimately detected by fluorescence spectroscopy. In this example, the biomolecule to be detected (i.e., the protein), and the specific binding pair that is detected (i.e., biotin-dye labeled streptavidin) are linked through another specific binding pair (i.e., primary and secondary antibodies).

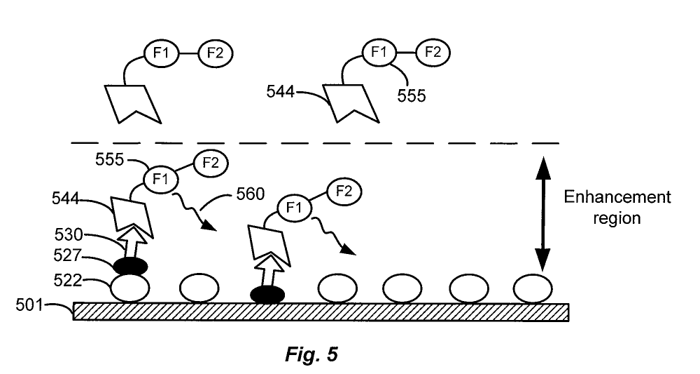

In FIG. 5, the substrate 501 comprises a plurality of metal colloidal aggregates 522 disposed thereon and further comprises a biomolecule 527 located on, or proximate to a metal colloidal aggregate 522. Alternatively, the biomolecule 527 is located on the substrate and the metal colloidal aggregates 522 are located proximate thereto. The first member of the specific binding pair 530 is added to the sample and binds to the biomolecule 527. Next, the complementary member of the binding pair 544, having two fluorescent groups 555 (F1 and F2) attached thereto is added to the sample. In the complementary member of the specific binding group, the fluorescence emission of F1 is quenched by processes of energy transfer to F2 (an acceptor fluorophore). Upon binding to the first member of the specific binding pair, F1 and F2 are brought within sufficient distance to a metal colloidal aggregate such that upon irradiation, the coupling between the F1 and F2 is disrupted, leading to a strong increase in the fluorescence emission (see arrow 560) of F1. Alternatively, F2 could also be a non-fluorescent group that quenches the fluorescence emission of F1.

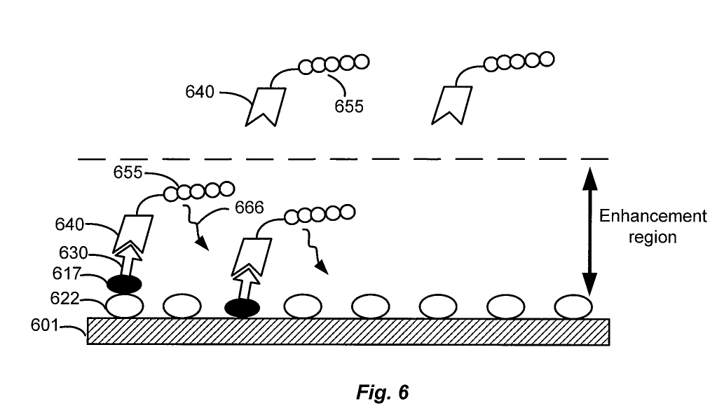

In FIG. 6, the substrate 601 comprises a plurality of metal colloidal aggregates 622 disposed thereon and further comprises a biomolecule 617 located on, or proximate to a metal colloidal aggregate 622. Alternatively, the biomolecule 617 is disposed on the substrate 601 and a plurality of metal colloidal aggregates 622 are located proximate thereto. The first member of the specific binding pair 630 is added to the sample and binds to the biomolecule 617. Next, the complementary member of the binding pair 640, having a plurality of fluorescent groups 655 (F1) attached thereto is added to the sample. The presence of a plurality of fluorescent groups (F1) on the complementary binding pair leads to a strong self-quenching effects due to the spatial density of fluorophores. Upon binding to the first member of the specific binding pair, the plurality of fluorophores are brought within sufficient distance to a metal colloidal aggregate such that upon irradiation, the self-quenching effects of the fluorophores are canceled, leading to a strong increase in the fluorescence emission (see arrow 666) of the individual fluorophores (see, Lakowicz, J. R., et al., Anal Biochem, 2003, 320, 13-20).

Optionally, in other embodiments, in FIGS. 5 and 6, the biomolecule is the first member of a specific binding pair, thereby obviating the requirement of adding a first member of the specific binding pair in a separate step.

In particular, the methods of the present invention for detecting a specific binding pair are particularly useful in immunoassays (e.g., western blot, ELISA, FLISA, and the like), hybridization assays (e.g., molecular beacon assays, Southern blot, northern blot), cell-based assays, microarrays (e.g., DNA arrays, RNA arrays, protein arrays, tissue arrays, antibody array), and the like. A description of assays that are suitable for the method of the invention and techniques for performing the assays is disclosed in U.S. Publication No. 2002/0160400 (see, paragraphs [0087]-[0099] therein), the disclosure of which is incorporated herein by reference. Additionally, methods for hybridization of nucleic acids are described in Nonradioactive In Situ Hybridization Application Manual by Roche Applied Science, 3rd ed (2002), which is incorporated herein by reference in its entirety. In one embodiment, the methods of the present invention are used to detect specific binding pairs in an assay selected from the group consisting of western blot, northern blot, Southern blot, cell-based assays, molecular beacon assays, protein arrays, DNA arrays and antibody arrays.

Monitoring, detecting an quantifying fluorescence is accomplished using common techniques known in the art (see, Lakowicz, J. R., Principles in Fluorescence Spectroscopy, Plenum Publishers, 1999, which is incorporated herein in its entirety). Suitable light sources include, for example, thermal emitters, light emitting diodes, and lasers, photomultipliers and photo semiconductors.

In certain aspect, metal enhanced fluorescence (MEF) as used herein is a ‘drop-in’ technology compatible with standard ELISA protocols. Colloidal aggregates are simply placed into the well before, during or after the assay is performed and before detection. The colloidal aggregates achieve signal enhancement and improve limits of detection (LOD) compared to existing enzyme linked immunosorbent assay protocols. In certain embodiments, lower limits of detection are possible using MEF with fluorescent dyes (e.g., near-IR dyes) because the desired signal is enhanced more than the undesired background. Improved limits of detection allow for reduced amounts of both sample and antibody, resulting in lower cost immunoassays and improved data quality.

In other instances, the MEF is accomplished by “in situ” formation of the colloidal metal aggregates. In these instances, the colloidal metal aggregates are formed in the systems as needed by introducing a colloidal metal and an aggregating agent, such as a salt. The colloidal metal aggregates form in situ. In still other instances, the colloidal metal aggregates are disposed on the substrates prior to assay performance. All such embodiments are within the scope of the present invention.

In certain other aspects, the present invention provides a two-color (or more) immunosorbent assay in standard ELISA format. In one instance, two-color assays provide for an internal standard by detecting a “housekeeping” protein in one channel and a target protein in a second channel. Having an internal standard on the same plate increases quantitative accuracy by reducing inter-experiment related errors since both signals are read on the same plate. Two-color immunosorbent assays will allow for direct ratiometric analysis of target proteins, reducing sample volume and increasing the amount of data generated per well addresses the ever-growing demand for high throughput technology for drug discovery, basic research, and clinical diagnostics.

As such, the present invention provides a two color ELISA (e.g., a near-infrared FLISA) method, comprising:
i) providing a substrate with at least two first specific binding members disposed thereon, wherein the at least two first specific binding members are the same or different;
ii) contacting the substrate with a sample containing at least two second specific binding members to form at least two specific bound pairs, wherein each of the at least two second specific binding members contain a first and a second fluorescent group, respectively; and
iii) irradiating the first and second fluorescent groups with excitation radiation to detect two color fluorescence.

A skilled artisan will appreciate that the methods herein are useful in a variety of assay formats including ELISA/FLISA, wherein the protein is coated or the virus is coated on the plate first. Direct/indirect/sandwich/and competitive assays are all within the scope of the present invention.

In certain aspects, the at least two first specific binding members (or more) are different. For example, an array of 2, 4, 8, 10, 20, 50, 100, 1000 or more first specific binding members, wherein all members are the same, different or mixtures can be disposed on the substrate.

In certain instances, attachment of the first and second fluorescent groups to each of the at least two second specific binding members is preformed prior to binding (e.g., step ii). In fact, in certain instances, the first and the second fluorescent groups are attached to each of the at least two second specific binding members, respectively by complementary binding members such as for example, biotin and streptavidin. In one embodiment, the two color (or more) fluorescence is detected simultaneous. In certain other aspects, the methods comprises iv) providing at least one metal colloidal aggregate proximate to the at least two specific bound pairs. In another aspect, the method provides incubating the assay components (e.g., prior to detection) with a protease (e.g., trypsin) to free the first and the second fluorescent groups.

In certain embodiments, a plurality of antibodies (2, 4, 8, 16, or more) are coated on a surface and one or all antibody interactions are detected. The two-color detection allows for higher throughput and/or provides an internal control. The applications of two-color can be done for in-cell or on-cell ELISAs, or for virus coat protein detection.

In another embodiment, the present invention provides a kit with at least one container, preferably 2 containers, wherein the first container comprises a salt and a protease and the second container comprises metal colloids. The kit optionally contains instructions for use.

The following examples are presented for the purpose of illustrating certain aspects of the invention and do not limit the scope of the invention in any way.

### V. Examples

### Example 1

### Metal Enhanced Fluorescence of IRDye® 800CW or Alexafluor® 680 with Silver Metal Colloidal Aggregates Over Dye Alone

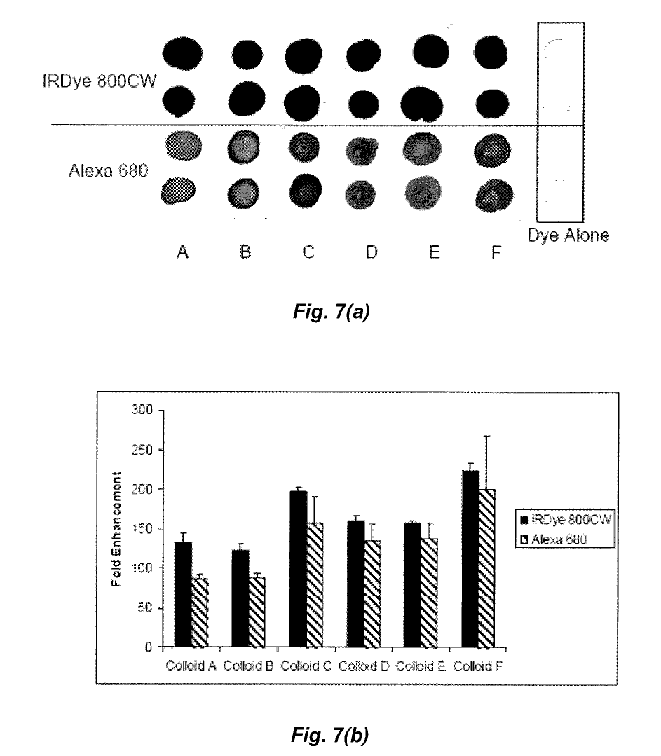

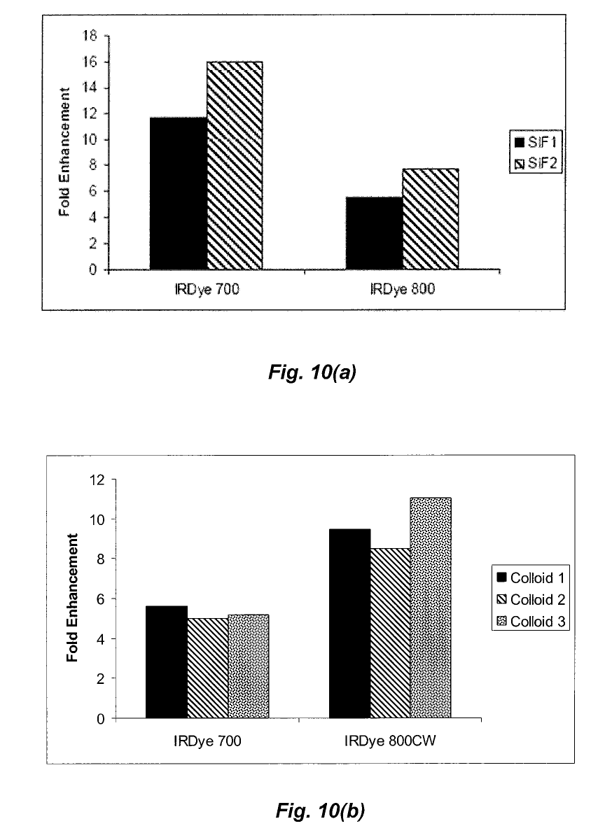

FIG. 7a shows various colloid preparations prepared by concentrating silver colloids and initiating controlled aggregation on the concentrated colloids by mixing with 0.1× phosphate buffered saline, spotted on glass. Each colloidal aggregate preparation was then spotted (in duplicates) with either IRDye® 800CW or Alexafluor® 680 labeled streptavidin. Dye-labeled streptavidin was also spotted alone (in duplicates) without the presence of colloid. The dye-spotted mixtures were then irradiated at the excitation wavelength of either IRDye® 800CW or Alexafluor® 680 and the fluorescence emission was recorded. Fluorescence enhancement of IRDye® 800CW or Alexafluor® 680 in the presence of a colloidal aggregate over each dye alone was graphed and is shown in FIG. 7b. (Fluorescence Enhancement=Integrated intensity of a colloid/dye mix divided by the integrated intensity of the dye alone). The best enhancement was provided the Colloid F sample with IRDye® 800CW, which gave a fluorescence enhancement of nearly 250-fold over dye alone. By comparison, silver island films and colloid coated surfaces with IRDye® 800CW gave enhancements of only 5 to 20-fold over dye alone (FIG. 10(a)). The fluorescence enhancement provided by the inventive metal colloidal aggregates are clearly superior over other silver island films and colloid coated surfaces known in the art.

### Example 2

### Metal Enhanced Fluorescence of IRDye® 800CW on Plain Glass and Silaniated Glass

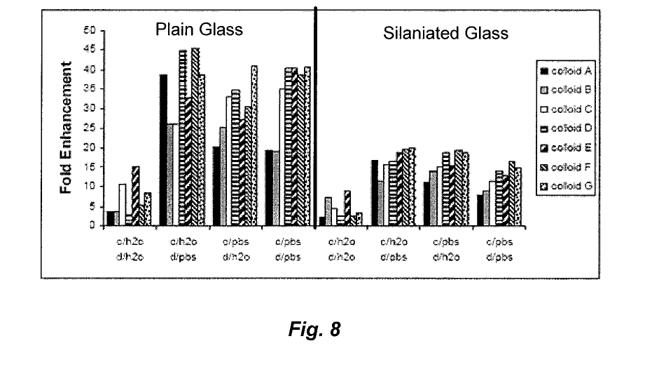

FIG. 8 is a bar graph that shows the fluorescence enhancement of IRDye® 800CW labeled streptavidin that is spotted over colloid mixtures which have been dried on glass slides. The results show that if a salt solution (0.1×PBS in this instance) is not used for the preparation of a colloidal aggregate nor is it present in the dye-labeled streptavidin solution that is spotted on a colloid aggregate, then fluorescence enhancement of the resultant dye/colloid mixture is only about 5-10 fold over the dye alone (see, FIG. 8 in the column where the x-axis is labeled cH₂O-dH₂O). However if the salt solution, i.e., 0.1×PBS, is added, either in the dye solution or colloid aggregate, then fluorescence enhancement is increased dramatically, on the order of about 25 to 45-fold on plain glass and from 15 to 25-fold on silaniated glass. The x-axis in the chart in FIG. 8 indicates whether a colloid aggregate and/or dye mixture contained salt: c=colloid; d=IRDye® 800CW; pbs=0.1×PBS added; H₂O=only water added/no salt.

### Example 3

### Metal Enhanced Fluorescence of IRDye® 800CW on Membrane with Silver Metal Colloidal Aggregates Over Dye Alone

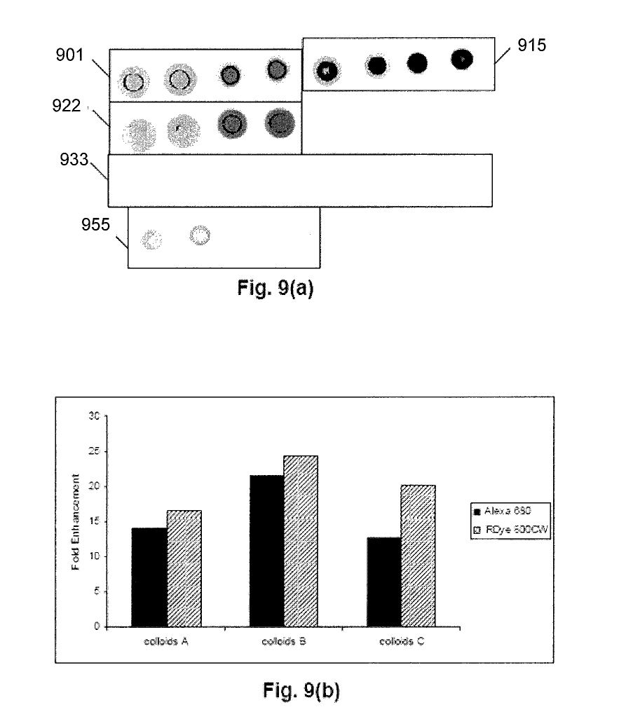

FIG. 9A shows various colloid preparations prepared by concentrating silver colloids and doing a controlled aggregation on the concentrated colloids by mixing them with 0.1× phosphate buffered saline, and then spotted on nitrocellulose membrane. Each colloidal aggregate preparation was then spotted (in duplicates) with either IRDye® 800CW or Alexa Fluor® 680 labeled strepavidin (901=Colloid A; 915=Colloid B; 922=Colloid F; 933=colloid aggregates alone). Dye-labeled streptavidin was also spotted alone 955 (in duplicates) without the presence of colloid preparation. The dye-spotted mixtures were then irradiated at the excitation wavelength of either IRDye® 800CW or Alexa Fluor® 680 and the fluorescence emission was monitored. Fluorescence enhancement of IRDye® 800CW or Alexa Fluor® 680 in the presence of a colloidal aggregate over each dye alone was graphed and is shown in FIG. 9B. This shows that metal colloidal aggregates deposited on nitrocellulose membrane is effective at enhancing fluorescence emission. In contrast, using silver island films and colloids on membranes with IRDye® 800CW or Alexa Fluor® 680 has produced no enhancement effects.

### Example 4

### Preparation of Silver Metal Colloidal Aggregates

Silver nitrate (AgNO₃; 250 mg) was added to a stirred solution of deionized water (dH₂O) (245 mL). To the stirring solution was added sodium citrate tribasic dehydrate (537 mg), followed by the addition of 5 mL of 0.5N solution of NaOH, and 250 mL of a 14 mM solution of ascorbic acid. The resultant silver colloid solution was centrifuged (30 min at 15000 g) and washed with dH₂O. The washed colloids are centrifuged again and re-suspended in 20 mL of dH₂O. To this concentrated colloidal solution is then gently mixed with an equal volume of 0.1×PBS to form a aggregated colloidal product.

FIG. 10(a) is a chart showing the metal enhanced fluorescence of IRDye® 800CW or IRDye® 700 labeled streptavidin on a silver island film coated glass substrate, known in the art, over dye alone. FIG. 10(b) is a chart showing the metal enhanced fluorescence of IRDye® 800CW or IRDye® 700 labeled streptavidin on a silver colloid coated glass substrate, known in the art, over dye alone.

### Example 5

### Single Color Near-IR FLISA

This example demonstrates sensitive near-IR FLISA separately at two different near-IR wavelengths. In this example, epidermal growth factor (EGF), IL-1 beta and IL-8 were assayed. The near-IR FLISA is detected by incubating with either streptavidin-IRDye® 800CW (SA-800CW, with fluorescence emission at ˜800 nm) or streptavidin-IRDye® 680 (SA-680, with fluorescence emission at ˜700 nm) commercially available from LI-COR. Washing the plate removes unbound labeled streptavidin, and the plates are thereafter imaged with an Aerius instrument (LI-COR, Inc.).

The background wells are treated exactly as all test wells, but no analyte is added. The background wells are replicated 16 times to estimate the background mean and standard deviation. A series of eight standard dilutions are added to each plate and the LODs are determined from the average of these replicates (Table 1). The LOD is defined to be the smallest concentration of a sample that can be reliably detected, producing a signal that is three times larger than the standard deviation of the system background. Table 1 below shows the limit of detection (LOD) values (pg/ml), at 3σ (99% confidence level) above background determined by near-IR FLISA. The data was all fit with a weighted (1/Y²) first order polynomial model.

TABLE 1
Limits of Detection

| | Colorimetric | | | | Near-IR FLISA with | |
|---|---|---|---|---|---|---|
| Target | SA-HRP | | Near-IR FLISA | | Silver Colloids | |
| | 800 Channel | | | | | |
| | | | SA-800CW | | SA-800CW | |
| | LOD pg/mL | R² | LOD pg/mL | R² | LOD pg/mL | R² |
| hEGF | 2 | 0.99 | 4 | 0.96 | 1 | 0.99 |
| | (N = 1) | | (N = 1) | | (N = 1) | |
| hIL-1 β | 2-5 | 0.91 | 1-13 | 0.93 | 5-8 | 0.94 |
| | (N = 2) | | (N = 5) | | (N = 4) | |
| hIL-8 | 5-21 | 0.92 | 5-81 | 0.64 | 1-36 | 0.88 |
| | (N = 5) | | (N = 7) | | (N = 7) | |
| | 700 Channel | | | | | |
| | | | SA-680 | | SA-680 | |
| | LOD pg/mL | R² | LOD pg/mL | R² | LOD pg/mL | R² |
| hEGF | 2 | 0.99 | 6 | 1.00 | 4 | 1.00 |
| | (N = 1) | | (N = 1) | | (N = 1) | |
| hIL-1 β | 2-5 | 0.91 | 3 | 0.99 | 1 | 0.99 |
| | (N = 2) | | (N = 1) | | (N = 1) | |
| hIL-8 | 5-21 | 0.92 | 27 | 0.40 | 4 | 0.86 |
| | (N = 5) | | (N = 1) | | (N = 1) | |

### Example 6

### Fractionation Enhancement

This example demonstrates size fractionation of silver colloid aggregates by centrifugation and the effect of size on enhancement of the near-IR dyes. Isolated fractions, which varied 3-fold in diameter were analyzed. The fractions were used in a biotinylated BSA/streptavidin-IRDye® 800CW assay format. All fractions gave some enhancement.

A 1.5 mL of a silver colloid nanoparticle preparation was added to a 12.5 mL glycerol step gradient (20-80%) and centrifuged for 20 min at 4000 rpm. A) 1 mL fractions were withdrawn from the top and the effective diameter was measured on the 90Plus Particle Size Analyzer (Brookhaven Instrument Corp). B) Following extensive washing, the fractions were then tested on a format binding assay. In this assay, biotinylated BSA was adsorbed to the plate, blocked with Odyssey Blocking Buffer (LI-COR), and incubated with streptavidin-IRDye® 800CW. The washed colloid fractions were added to the respective wells in 0.05×PBS, shaken for 5 min at room temp in the dark and imaged on the Aerius. Background wells were incubated with streptavidin-IRDye® 800CW, but no silver colloids were added.

### Example 7

### MEF on Near-IR Antibody Arrays and Cellular Assays

This example demonstrates that the colloidal aggregates can be used in an array format.

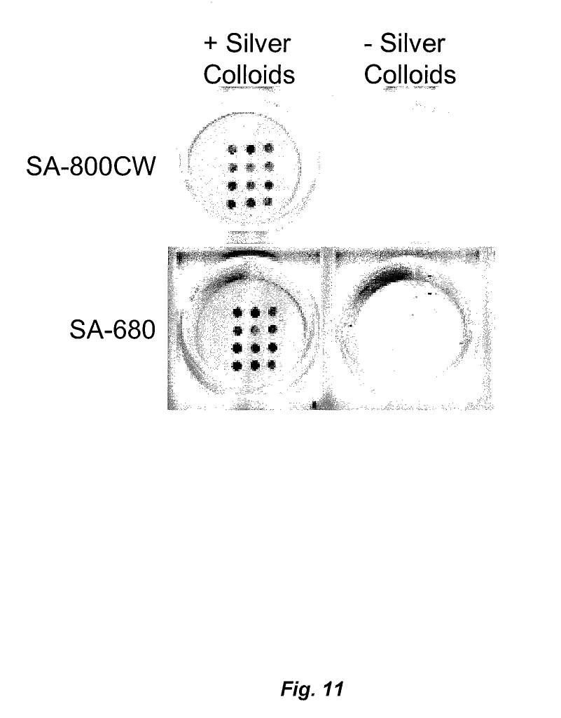

In this example, silver colloid aggregates were utilized in multi-well antibody arrays (commerically available from Quansys Biosciences) to determine the amount of enhancement. Antibodies to 12 different cytokines were applied (nL volumes) to each well in a 12-plex array. The plate was treated with a mixture of the cytokine targets, followed by a mixture of biotinylated cytokine antibodies, and finally IRDye-streptavidin (either SA-800CW or SA-680) (FIG. 11). Analysis of these images gave a SNR that was 15-fold higher in the wells containing silver colloidal aggregates.

Silver colloid nanoparticles were also applied to an in-cell Western assay and a 15-20 fold enhancement in signal was observed. In this assay, a ligand labeled with IRDye® 800CW was applied to fixed cells and fluorescent signal from each well was quantified.

### Example 8

### A Homogeneous Assay Using Colloidal Aggregates

In this homogeneous assay, after an ELISA assay is performed, but prior to detection, a solution containing trypsin is added to liberate the dye into the solution. The colloids are added to the plate, incubated for 5 min with an aggregating agent (e.g., salt) to form the aggregates and then detected. With the dye and the colloidal aggregates forming a homogeneous mixture, the enhancement is actually increased over using the same colloids with the dye attached to the surface of the plate.

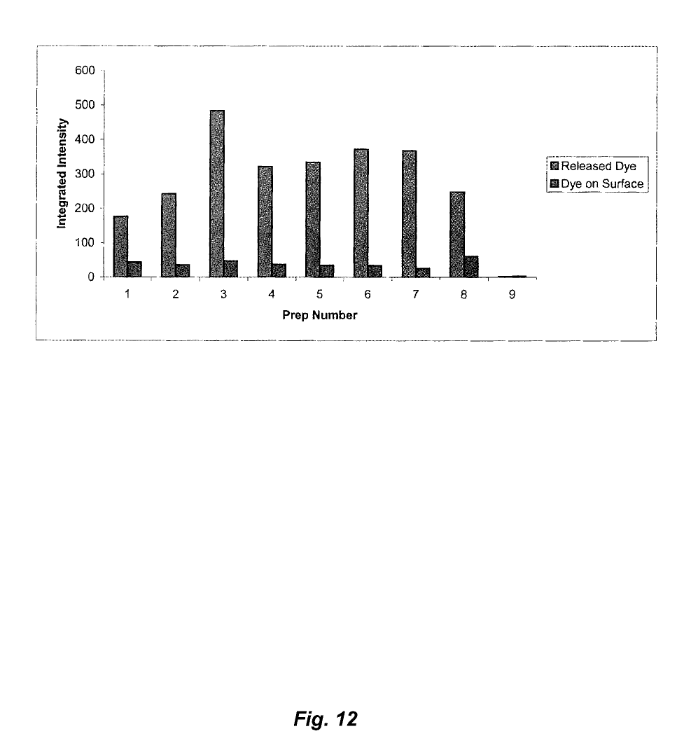

FIG. 12 shows the results of the experiment. As shown therein, Samples 1 through 8 are each different silver colloid preps being tested. Sample 9 contains no colloids of the present invention. To the wells, a PBS salt solution either with trypsin (released dye) or without trypsin (dye on surface) was added. The trypsin will cleave proteins and release the IRDye® 800CW into solution. Then to rows 1-8 colloids were added (water was added to row 9). The samples were incubated for 5 min and scanned on a Aerius imager.

### Example 9

### A FLISA Test Reaction

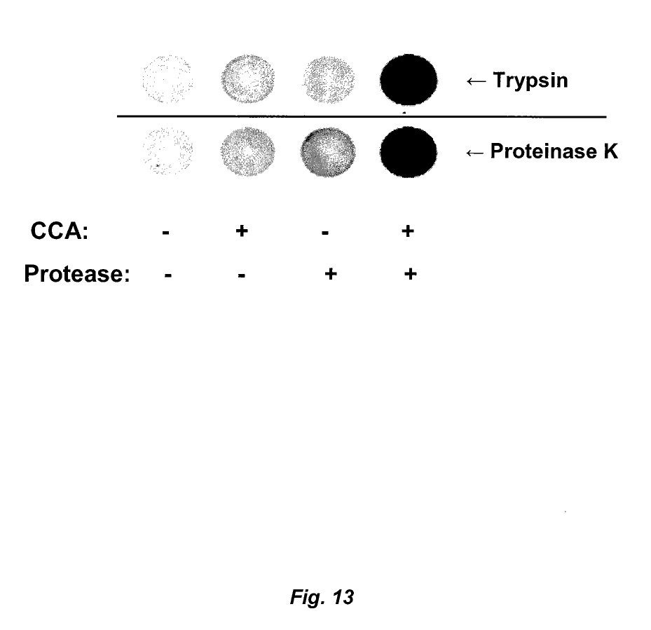

FIG. 13 shows an image of a FLISA test reaction (using Biotinylated BSA as the sample and IRDye® 800CW labeled streptavidin for detection). All wells contain Biotinylated BSA and Streptavidin-IRDye® 800CW. Buffer alone was added to samples in column 1; A low concentration of metal colloidal aggregates were added to samples in column 2; protease (either trypsin or proteinase K as shown) was added to samples in column 3; and both a low concentration of metal colloidal aggregates and protease were added to the samples in column 4.

### Example 10

### Limit of Detection Experiment

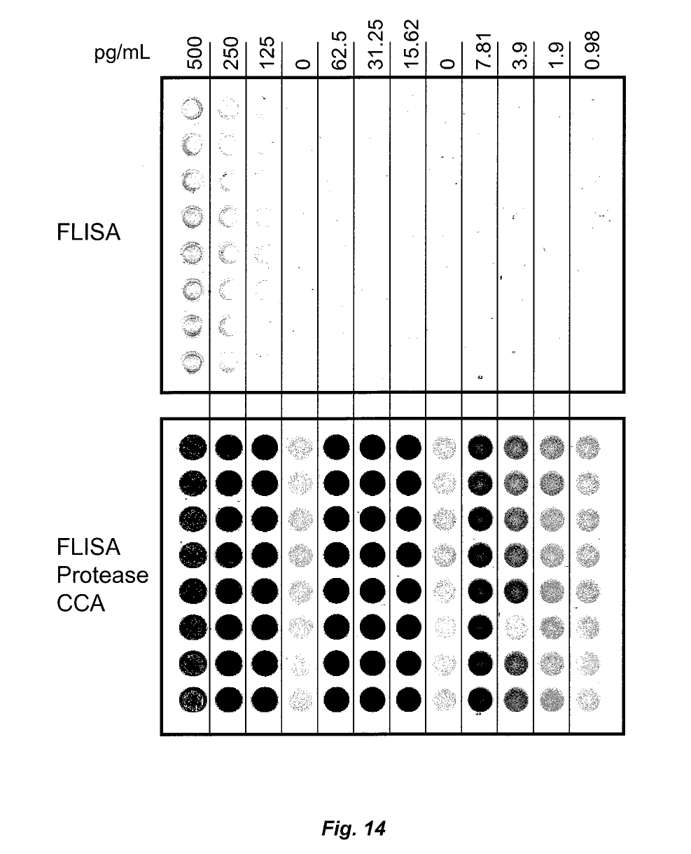

FIG. 14 shows a Limit of Detection experiment wherein FLISA tests were done for IL-8. Two-fold dilutions of IL-8 were samples as indicated (each column contains a specific concentration of IL-8, with two columns containing no IL-8). The plate was prepared for a normal FLISA reaction, according to the manufacture's instructions. The plate was imaged on an Aerius imager (top image). Protease was then added to the plate and the plate was incubated for 30 min at 37° C. Following the incubation, metal colloidal aggregates were added to the samples and the plate was rescanned on the Aerius imager. For this plate—the LOD for IL-8 without using metal colloidal aggregates was 27.19 pg/ml. The LOD for IL-8 using protease and metal colloidal aggregates was 1.12 pg/ml.

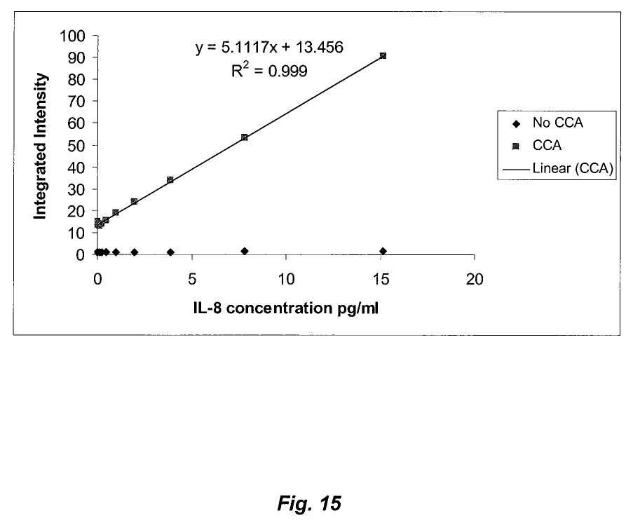

FIG. 15 shows an integrated intensity verses the concentration of IL-8 with and without using protease and metal colloidal aggregates. A line fit is also shown for the metal colloidal aggregate samples.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A system for detecting a specific binding pair, said system comprising:
a substrate having a first member of a specific binding pair disposed thereon;
a second member of the specific binding pair, wherein at least one member of the binding pair comprises a fluorescent group;
at least one metal colloidal aggregate proximate to said first binding member;
a light source for irradiating said fluorescent group at its excitation wavelength; and
a detector configured for monitoring emission from said fluorescent group.

2. The system of claim 1, wherein said substrate is selected from the group consisting of glass, silanized glass, a quartz plate, plastic, a carbon nanotube, a metal and a membrane.

3. The system of claim 2, wherein said substrate is selected from the group consisting of glass, silanized glass, a nylon membrane, a polyvinylidine difluoride (PVDF) membrane, a nitrocellulose membrane, polypropylene, polystyrene, polyvinylchloride and polycarbonate.

4. The system of claim 1, wherein said metal is selected from the group consisting of silver, gold, aluminum, rhenium, ruthenium, rhodium, palladium, silver, copper, osmium, iridium, platinum and a combination thereof.

5. The system of claim 4, wherein said at least one metal is selected from the group consisting of silver, gold and aluminum.

6. The system of claim 1, wherein said at least one metal colloidal aggregate has a diameter of between 50 nm and 3 μm.

7. The system of claim 6, wherein said metal colloidal aggregate has a diameter of between 250 nm and 2 μm.

8. The system of claim 7, wherein said at least one metal colloidal aggregate has a diameter of between 500 nm and 1.5 μm.

9. The system of claim 1, wherein said colloidal aggregate is coated with a member selected from the group consisting of a polymer, a gel, an adhesive, an oxide, SiO₂, mercaptan, casein, BSA, nucleic acid, and a biologic material.

10. The system of claim 9, wherein said metal colloidal aggregate is coated with SiO₂.

11. The system of claim 1, wherein said metal colloidal aggregate comprises an aggregating agent.

12. The system of claim 11, wherein said aggregating agent is a salt.

13. The system of claim 12, wherein said salt is a member selected from the group consisting of an alkali metal salt, an alkaline metal salt, an organic salt, a transition metal salt and a combination thereof.

14. The system of claim 12, wherein said salt is selected from the group consisting of NaCl, KCl, Na₂HPO₄, NaH₂PO₄, KH₂PO₄, LiCl, tris(hydroxymethyl)aminomethane hydrochloride, and combinations thereof.

15. The system of claim 12, wherein said salt is a biological buffer system selected from the group consisting of phosphate buffer, saline, and Tris-HCl.

16. The system of claim 1, wherein said first member of said specific binding pair is one member of the pairs selected from the group consisting of a protein/antibody, DNA/cDNA, RNA/cRNA, antigen/antibody, secondary antibody/antibody, peptide nucleic acid/complementary strand, protein/carbohydrate, drug/drug receptor, toxin/toxin receptor, carbohydrate/lectin or carbohydrate receptor, peptide/peptide receptor, protein/protein receptor, peptide/small molecule, nucleic acid/protein, nucleic acid/enzyme, receptor/small molecule, enzyme/substrate, DNAzyme/substrate, RNAzyme/substrate, protein/protein, aptmer/ligand, hormone/hormone receptor, ion/chelator, biotin/avidin, biotin/streptavidin, biotin/neutravidin, folate/folate-binding protein and IgG/protein A or protein G.

17. The system of claim 1, wherein said first member comprises a fluorescent group.

18. The system of claim 17, wherein said fluorescent group is a near-infrared fluorophore that emits in the range of between 650 and 900 nm.

19. The system of claim 17, wherein said fluorescent group is selected from the group consisting of polymethine dyes, pthalocyanine dyes, cyanine dyes, xanthene dyes, fluorine dyes, rhodamine dyes, coumarin dyes, fluorescein dyes and dipyrromethene boron difluoride dyes.

20. The system of claim 18, wherein said near-infrared fluorophore is selected from the group consisting of polymethine dyes, pthalocyanine dyes and cyanine dyes.

21. The system of claim 20, wherein said near-infrared fluorophore is a cyanine dye.

22. (canceled)

23. The system of claim 1, wherein said second member of the specific binding pair comprises a fluorescent group.

24. The system of claim 23, wherein said fluorescent group is a near-infrared fluorophore that emits in the range of between 650 and 900 nm.

25. The system of claim 23, wherein said fluorescent group is selected from the group consisting of polymethine dyes, pthalocyanine dyes, cyanine dyes, xanthene dyes, fluorine dyes, rhodamine dyes, coumarin dyes, fluorescein dyes and dipyrromethene boron difluoride dyes.

26. The system of claim 24, wherein said near-infrared fluorophore is selected from the group consisting of polymethine dyes, pthalocyanine dyes and cyanine dyes.

27. The system of claim 26, wherein said near-infrared fluorophore is a cyanine dye.

28. The system of claim 23, wherein binding of said first member of the specific binding pair to said second member of said binding pair brings the fluorophore within a sufficient distance of said metal colloidal aggregate to enhance fluorescence.

29. The system of claim 28, wherein said sufficient distance is between about 10 Å to about 1000 Å.

30. The system of claim 29, wherein said sufficient distance is between about 25 Å to about 1000 Å.

31. The system of claim 30, wherein said sufficient distance is between about 50 Å to about 200 Å.

32. The system of claim 1, wherein said specific binding pair is a plurality of specific binding pairs, wherein each specific binding pair may be the same or different.

33. The system of claim 1, wherein said at least one metal colloidal aggregate proximate to said first binding member is disposed on said substrate.

34. A method for detecting a specific binding pair in a sample, said method comprising:
i) providing a system of claim 1, which system comprises a substrate having at least one biomolecule disposed thereon and at least one metal colloidal aggregate proximate thereto;
ii) contacting the substrate with a first member of a specific binding pair wherein the first member of the specific binding pair attaches to the biomolecule;
iii) contacting the substrate with a sample containing a second member of the specific binding pair; wherein the second member of a specific binding pair comprises at least one fluorescent group attached thereto; and
iv) irradiating the at least one fluorescent group with excitation radiation to detect the fluorescent group.

35. A method for detecting a specific binding pair in a sample, said method comprising:
i) providing a system of claim 1, which system comprises a substrate having a first member of a specific binding pair disposed therein and at least one metal colloidal aggregate proximate thereto;
ii) contacting the substrate with said sample containing a second member of the specific binding pair, wherein said second member of a specific binding pair comprises at least one fluorescent group attached thereto; and
iii) irradiating said at least one fluorescent group with excitation radiation to detect fluorescence.

36. A method for detecting a specific binding pair in a sample, said method comprising:
i) providing a substrate having at least one biomolecule disposed thereon and at least one metal colloidal aggregate proximate thereto;
ii) contacting the substrate with a first member of a specific binding pair wherein the first member of the specific binding pair attaches to the biomolecule;
iii) contacting the substrate with a sample containing a second member of the specific binding pair; wherein the second member of a specific binding pair comprises at least one enzyme group attached thereto; and
iv) incubating the substrate with a chemiluminescent compound to detect a luminescent product.

37. A method for detecting a specific binding pair in a sample, said method comprising:
i) providing a substrate having a first member of a specific binding pair disposed thereon and at least one metal colloidal aggregate proximate thereto;
ii) contacting the substrate with a sample containing a second member of the specific binding pair, wherein the second member of a specific binding pair comprises at least one enzyme group attached thereto; and
iii) incubating the substrate with a chemiluminescent compound to detect a luminescent product.

38. The method of claim 37, wherein said method is used to detect a complimentary binding pair in an assay selected from the group consisting of western blot, northern blot, Southern blot, cell-based assays, molecular beacon assays, protein arrays, DNA arrays and antibody arrays.

39. A two color near-infrared FLISA method, said method comprising:
i) providing a substrate with at least two first specific binding members disposed thereon, wherein said at least two first specific binding members are the same or different;
ii) contacting said substrate with a sample containing at least two second specific binding members to form at least two specific bound pairs, wherein each of said at least two second specific binding members contain a first and a second fluorescent group, respectively; and
iii) irradiating said first and second fluorescent groups with excitation radiation to detect two color fluorescence.

40. The method of claim 39, wherein said at least two first specific binding members are different.

41. The method of claim 39, wherein attachment of said first and second fluorescent groups to each of said at least two second specific binding members is performed prior to step ii.

42. The method of claim 41, wherein said first and second fluorescent groups are attached to each of said at least two second specific binding members, respectively by complementary binding members.

43. The method of claim 39, wherein said two color fluorescence is detected simultaneously.

44. The method of claim 39, further comprising iv) providing at least one metal colloidal aggregate proximate to said at least two specific bound pairs.

45. The method of claim 39, wherein prior to step iv), incubating with a protease to free said first and said second fluorescent groups.

46. The method of claim 44, wherein said metal colloidal aggregate is coated.

47. The system of claim 1, wherein said at least one colloidal aggregate is a plurality of colloidal aggregates.

48. The system of claim 47, wherein said plurality of colloidal aggregates are randomly disposed on said substrate.

49. The system of claim 1, wherein said at least one dye is homogenously mixed with said at least one metal colloidal aggregate prior to being disposed on said substrate.

50. The system of claim 17, wherein said fluorescent group is within a sufficient distance of said metal colloidal aggregate to enhance fluorescence.

51. The system of claim 50, wherein said sufficient distance is between about 10 Å to about 1000 Å.

52. The system of claim 50, wherein said sufficient distance is between about 25 Å to about 1000 Å.

53. The system of claim 50, wherein said sufficient distance is between about 50 Å to about 200 Å
